(19) United States
(12) Patent Application Publication
(10) Pub. No.: US 2008/0044441 A1
(43) Pub. Date: Feb. 21, 2008

(54) Mixed haptenized tumor cells and extracts and methods of treating or screening for cancer

(75) Inventor: David Berd, Wyncote, PA (US)

Correspondence Address: MORGAN, LEWIS & BOCKIUS LLP, 1701 MARKET STREET, PHILADELPHIA, PA, 19103-2921, US

(73) Assignee: Thomas Jefferson University

(21) Appl. No.: 11/643,626

(22) Filed: Dec. 20, 2006

Related U.S. Application Data
(63) Continuation of application No. 10/356,887, filed on Feb. 3, 2003, now abandoned.
(60) Provisional application No. 60/353,769, filed on Feb. 1, 2002.

(51) Int. Cl. A61K 39/385; A61P 35/00; C12N 5/02

(52) U.S. Cl. 424/277.1; 435/325

(57) ABSTRACT

This invention relates to compositions comprising multi-haptenized tumor cells and extracts thereof, methods for preparing the compositions, vaccines comprising such multi-haptenized tumor cells, and methods for treating cancer with such vaccines. In a specific embodiment, melanoma and ovarian adenocarcinoma cells are multi-haptenized, wherein the tumor cells are differentially haptenized either with a dinitrophenyl group coupled to ε-amino groups, or with a sulfanilic acid group coupled to aromatic side chains of histidine and tyrosine. A method of SA-haptenization is also provided.

This application claims priority from U.S. Provisional Application Ser. No. 60/353,769, filed Feb. 1, 2002, which is hereby incorporated by reference in its entirety.

### FIELD OF THE INVENTION

The invention relates to compositions comprising mixed preparation multi-haptenized tumor cells or cell extracts, methods for preparing the compositions, vaccines comprising such compositions, and methods for treating cancer with such vaccines.

### BACKGROUND OF THE INVENTION

### Haptenized Tumor Cell Vaccines

An autologous whole-cell vaccine modified with the hapten dinitrophenyl (DNP) has has been shown to produce inflammatory responses in metastatic sites of melanoma patients. The survival rates of patients receiving post-surgical adjuvant therapy with DNP-modified vaccine are markedly higher than those reported for patients treated with surgery alone. Intact or viable cells are preferred for the vaccine.

U.S. Pat. No. 5,290,551, discloses and claims vaccine compositions comprising haptenized melanoma cells. Melanoma patients who were treated with these cells developed a strong immune response. This response was detected, e.g., in a delayed-type hypersensitivity (DTH) response to haptenized and non-haptenized tumor cells. More importantly, the immune response increased the survival rates of melanoma patients.

It is known from animal studies that immunization of mice with syngeneic lymphocytes modified with arsanilic acid induces strong T cell responses against those modified cells, including DTH (Bach et al., J. Immunol., 1978;121:1460) and cytotoxic T cells (Sherman et al., J. Immunol., 1978; 121:1432). Injection of arsanilic acid into the rat kidney induced a brisk autoimmune nephritis (Rennke et al., Kidney International, 1994;45:1044). Obviously, the administration of even minute amounts of arsanilic acid into human is unacceptable, but sulfanilic acid, a non-toxic compound in small amounts, should induce a similar immunological effect (Nahas and Leskowitz, supra, 1980). Both compounds can be coupled to tyrosine and histidine after being diazotized by treatment with sodium nitrite. Moreover, immunization of animals with sulfanilic acid-modified protein can induce autoimmunity (Weigle, J. Exp. Med., 1965;122:1049). A third potentially interesting hapten in this category is phosphorylcholine (PC), in light of the work of Kim et al. (Eur. J. Immunol., 1992;22:775). However, it has not been established that these haptens will be effective in humans; on the contrary, Nahas and Leskowitz, supra, suggest otherwise.

Haptenized tumor cell vaccines have also been described for other types of cancers, including lung cancer, breast cancer, colon cancer, pancreatic cancer, ovarian cancer, and leukemia (see International Patent Publication Nos. WO 96/40173 and WO 00/09140; and U.S. Pat. No. 6,333,028, and the associated techniques and treatment regimens optimized (see International Patent Publication Nos. WO 00/38710, WO 00/31542, WO 99/56773, WO 99/52546, and WO 98/14206).

Generally, the immune response to haptenized cells has been found to be independent of the choice of hapten, but dependent on the functional group to which the hapten is attached. In particular, it has been reported that haptenization of ε-amino groups of lysine and —COOH groups of aspartic acid and glutamic acid is effective for a robust immune response, and that haptenization of aromatic groups (such as tryptophan) potentially results in a less effective or ineffective immune response (Nahas and Leskowitz, Cellular Immunol., 1980;54:241).

Various discoveries have improved the efficacy of haptenized tumor cell preparation vaccines. These include modification of the dosing schedule and number of cells per dose (see PCT Publication Nos. WO 99/40925 and WO 99/56773) and using an induction dose of tumor cells, either haptenized or not free of adjuvant, and lower doses of cells (see PCT Publication No. WO 01/56601).

Other attempts to improve haptenized tumor cell vaccines yielded less dramatic results. One such example includes haptenization of the same cell with haptens reactive with different functional groups (PCT Publication No. WO 00/38710).

These discoveries have led to rapid advances in the treatment of cancer, particularly melanoma, by immunotherapy. Nevertheless, there remains a need in the art for even more effective therapies, since the response rates achieved with the haptenized tumor cell vaccine technologies mentioned above, while impressive, have not reached 100%. There is also a need in the art for effective vaccines using fewer cells, e.g., fewer than about 10⁷ cells per dose. This is especially critical for the treatment of an early stage cancer, when the number of cells obtainable from a resected tumor may be fewer than necessary for vaccine preparation as described above.

The present invention addresses these and other needs in the art.

### SUMMARY OF THE INVENTION

Provided by the present invention is a composition comprising a mixture of at least a first and a second haptenized tumor cell preparation, wherein the haptenized tumor cell preparations are differentially haptenized, and originate from the same tumor type as the tumor type of a subject intended for treatment with the composition. The composition may comprise two different haptens attached to functional groups of polypeptides associated with the tumor cells. The functional groups can be, for example, amino groups, carboxylic groups, and aromatic groups. In one embodiment one hapten is sulfanilic acid (SA), and the second hapten is dinitrophenyl (DNP). Preferably, the numbers of cells in the first and second haptenized tumor cell preparations differ by no more than two-fold. More preferably, the numbers of cells in the first and second haptenized tumor cell preparations are about equal. The tumor cells may also have been rendered incapable of growth.

The present invention also provides for a vaccine for treating cancer comprising the composition described above and an adjuvant. Suitable adjuvants include, but are not limited to, BCG. The invention also provides for a method for treating cancer in a subject comprising administering such a vaccine to the subject. The subject may be a human.

The invention also provides for a method of preparing a composition for use in a cancer vaccine, which method comprises differentially haptenizing at least a first and a second fraction of a tumor cell preparation and mixing cells from the differentially haptenized fractions, wherein the tumor cell preparation originates from the same type of tumor as the tumor of a subject for whom the vaccine is intended. In one embodiment, the first fraction is haptenized with a first hapten, and the second fraction is haptenized with a second hapten. The first and second haptens may be conjugated to functional groups selected from amino groups, carboxylic acid groups, aromatic groups, hydroxyl groups, imidazole groups, and sulfhydryl groups. For example, the first hapten can be conjugated to an aromatic group and the second hapten conjugated to a primary amino group or a carboxylic acid group. In such a case, the first hapten can be sulfanilic acid (SA), and the second group dinitrophenyl (DNP). The numbers of cells mixed from the first and second fractions preferably differ by no more than two fold, and can, more preferably, be about equal. The tumor cells may also have been rendered incapable of growth.

The invention also provides for a method of haptenizing a cells with sulfanilic acid, comprising the steps of contacting an aromatic group of the cell with a sulfanilic-acid-diazonium-salt in a buffered solution at a pH lower than 8.2 to initiate a haptenization reaction, incubating the solution for less than 15 minutes, and terminating the haptenization reaction. In one embodiment, the pH is between 7.0 and 7.8, more preferably about 7.2. In another embodiment, the incubating step may be between 3 and 10 minutes, more preferably about 5 minutes. The buffered solution can be, for example, HBSS or PBS.

The invention will be further explained by the Drawings, Detailed Description, and Examples.

### BRIEF DESCRIPTION OF THE DRAWINGS

FIG. 1. This figure shows the effect of changing the pH during the haptenization process on the SA modification of melanoma cells. The positive control was SA-BSA and the negative controls were HSA and unmodified human melanoma cells (Unmod TC).

FIG. 2 This figure shows the effect of changing the length of the period of incubation during the haptenization process on the SA modification of melanoma cells. The positive control was SA-BSA and the negative controls were HSA and unmodified human melanoma cells (HOL TC-Unmodified).

FIG. 3 This figure shows the effect of buffered solution on the SA modification of tumor cells during the SA haptenization process run at pH 7.2 with an incubation time of 5 minutes. The positive control was SA-BSA and the negative controls were unmodified tumor cells (Unmod TC) and HSA. The buffered solutions, HBSS, PBS and BBS were tested.

### DETAILED DESCRIPTION OF THE INVENTION

The present invention provides a composition of haptenized tumor cell preparations, useful for preparing more effective vaccines for immunotherapy of cancer. Each haptenized tumor cell preparation comprises a mixture of differently haptenized tumor cells, i.e., different fractions of a preparation of the same type of tumor cells have been haptenized with different haptens, for example, dinitrophenyl in one fraction and sulfanilic acid in another. Mixing the two (or more) haptenized fractions together yields a composition of the invention, which, when administered with an adjuvant, provides a more effective cancer immunotherapy than administering either preparation alone, or a preparation of tumor cells bearing both haptens on the same cells.

The work described herein has provided strong support for the idea that immunizing patients with hapten-modified tumor cells can induce immunity to unmodified tumor cells. The present invention provides a rationale for achieving improved results in humans by the addition of a second hapten. More particularly, the invention permits the use of a decreased number of tumor cells in a vaccine, or a more effective immune response, or both. Furthermore, the immune response is, in a specific embodiment, enhanced by a method of haptenization previously believed to be ineffective. The invention also advantageously provides for the modification of tumor cells with the hapten sulfanilic acid (SA) wherein the yield of intact haptenized cells at the end of the haptenization process is increased. This invention permits the use of smaller starting cell samples in the creation of SA-haptenized tumor cells, thereby allowing the vaccine, containing SA-haptenized tumor cells, to be created from smaller tumors early in the progression of the cancer.

The present invention offers specific advantages. For example, haptenization of different functional groups on proteins on different tumor cells increases the immuno-poteniating effect. Such an approach also increases the number of patients amenable to this treatment by negating any predisposition of an individual to tolerate one or the other individual hapten(s). In addition, because the overall density of hapten groups can be greater, each haptenization reaction can be run under milder conditions, thereby better preserving cell integrity.

The various aspects of the invention will be set forth in greater detail in the following sections. These sections are intended to facilitate understanding the invention, and are in no way intended to be limiting thereof.

### DEFINITIONS

The following defined terms are used throughout the present specification, and should be helpful in understanding the scope and practice of the present invention.

As used herein, the term “tumor cell preparation” refers to haptenized intact tumor cells, which may be viable or not and which may include tumor cell lysate, whole cell tumor extracts or lysates, or debris, such as tumor cell membranes, from their preparation; tumor cell membranes; and tumor cell peptide extracts, each of which are described in greater detail below. Tumor cells useful for the present invention includes both cells which exclude and cells which do not exclude Trypan Blue.

“Haptenization” (and all grammatical forms thereof), means chemically derivatizing a tumor cell or tumor cell extract (e.g., membrane, whole cell lysate, or peptide) by reacting an amino acid functional group with a chemical entity. Specific side chain reactions and chemical entities for haptenization are described in greater detail below.

As used herein, a “bi-haptenized”, “multi-haptenized” or “mixed haptenized” tumor cell preparation means a composition comprising two or more tumor cell preparations, in which each tumor cell preparation is differently haptenized. “Bi” means two.

A “live” cell means a cell that has an intact cell, plasma, or “outer” membrane as assessed by exclusion of a supravital dye such as Trypan Blue. A live cell may be capable of growth or maintenance, and division or multiplication, or attenuated, i.e., incapable of division and multiplication. A cell can be rendered attenuated by, for example, irradiation.

“Dead” cells means cells that do not exclude supravital dyes such as Trypan Blue, propidium bromide, or ethidium bromide, as assessed in an exclusion experiment (see, e.g., Methods In Analysis Of Apoptosis And Cell Necrosis by Darzynkiewicz Z., In: The Purdue Cytometry CD-ROM Vol 3, J. Parker, C. Stewart, Guest Eds.; J. Paul Robinson, Publisher, Purdue University, West Lafayette, 1997). Dead cells are incapable of division or multiplication. A “dead” cell can be prepared by, e.g., ethanol treatment of a live cell. A dead cell may appear intact, e.g., by microscopic inspection, meaning that the cellular shape resembles that of a live cell. A “fixed” cell is one example of a dead cell.

A “lysed” cell is no longer intact, meaning that the cellular shape does not resemble that of a live cell.

The “total” number of tumor cells in a preparation means the sum of live and dead tumor cells in the preparation.

An “anti-tumor response” is at least one of the following: tumor necrosis, tumor regression, tumor inflammation, tumor infiltration by activated T lymphocytes, activation of tumor infiltrating lymphocytes, delayed-type hypersensitivity (DTH) response, or a clinical response. Clinical response criteria for anti-tumor response resulting from treatment according to the present invention include complete, partial, or mixed response, as well as stable disease. Other clinical responses that may be observed following the treatment of the invention is prolongation of time to relapse, or prolongation of survival.

A “vaccine composition” is a composition as set forth previously further comprising an adjuvant, including an immunostimulatory cytokine or lymphokine.

The terms “vaccine”, “immune therapy” and “immunotherapy” are used herein interchangeably to administration of a composition comprising a tumor cell preparation (preferably haptenized) to treat a cancer, e.g., after surgical resection of the tumor. “Efficacy of an immunotherapy” is the degree to which the immunotherapy elicits am anti-tumor response in an individual subject, or the percentage of subjects in which an anti-tumor response develops as a result of treatment. Preferably efficacy is determined by composition to controls that harbor the spontaneous tumor but receive either no therapy, sham therapy, or an alternative therapy.

The term “treat” means to attempt to elicit an anti-tumor response against cells of the tumor, i.e., the cancer. An anti-tumor response includes, but is not limited to, increased time of survival, inhibition of tumor metastasis, inhibition of tumor growth, tumor regression, and development of a delayed-type hypersensitivity (DTH) response to unmodified tumor cells.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

A “formulation” refers to an aqueous medium or solution for the preservation of haptenized tumor cells, which is preferably directly injectable into an organism. The aqueous medium can include salts or sugars, or both, at about an isotonic concentration.

The phrase “pharmaceutically acceptable” refers to molecular entities, at particular concentrations, and compositions that are physiologically tolerable and do not typically produce an allergic or similar untoward reaction, such as gastric upset, fever, dizziness and the like, when administered to a human or non-human animal. Preferably, as used herein, the term “pharmaceutically acceptable” means approved by a regulatory agency of the Federal or a state government or listed in the U.S. Pharmacopoeia or other generally recognized pharmacopoeia for use in humans or non-human animals.

As used herein, the term “isolated” means that the referenced material is removed from the natural environment in which it is normally found. In particular, an isolated biological material is free of cellular components. An isolated peptide may be associated with other proteins or nucleic acids, or both, with which it associates in the cell, or with cellular membranes if it is membrane-associated. An isolated organelle, cell, or tissue is removed from the anatomical site in which it is found in an organism. An isolated material may be, but need not be, purified.

The term “purified” as used herein refers to material that has been isolated under conditions that reduce or eliminate unrelated materials, i.e., contaminants. For example, a purified protein is preferably free of other proteins or nucleic acids with which it is associated in a cell; a purified cell is free of unrelated cells and tissue matrix components.

A “subject” is a human or a non-human animal who may receive haptenized tumor cells formulated in a composition of the invention. Preferably the subject is a human. However, the invention is also contemplated for veterinary medicine, particularly for treatment of domestic pets (dogs, cats), and livestock (horses, cows, pigs, etc.)

The term “differentially haptenized” as used herein refers to mixture of at least two haptenized tumor cells, wherein a first cell was haptenized under a particular condition or using a particular reagent and a second cell was haptenized under a different condition or using a different reagent. The conditions or reagents may differ so that, for example, different amino acids are haptenized on the proteins of the first and second tumor cells, and/or that the hapten attached to the first cell is different from the hapten attached to the second cell.

### Tumor Cells

The present invention is directed for use in the preparation of mixed-haptenized tumor cell vaccines for treating cancer, including metastatic and primary cancers. Cancers treatable with the present invention include solid tumors, including carcinomas, and non-solid tumors, including hematologic malignancies. Examples of solid tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Hematologic malignancies include leukemias, lymphomas, and multiple myelomas. The following are non-limiting preferred examples of the cancers treatable with the composition and methods of the present invention: melanoma, including stage-4 melanoma; ovarian, including advanced ovarian; leukemia, including but not limited to acute myelogenous leukemia; colon, including colon metastasized to liver; rectal, colorectal, breast, lung, kidney, and prostate cancers.

### Intact Tumor Cells

The compositions of the present invention are prepared from tumor cells, e.g., cells obtained from tumors surgically resected in the course of a cancer treatment regimen as described above. Tumor cells to be used in the present invention may be prepared as follows. Tumors are processed as described by Berd et al., Cancer Res., 1986;46:2572, Sato, et al., Cancer Invest., 1997;15:98, U.S. Pat. No. 5,290,551, and applications U.S. Ser. Nos. 08/203,004; 08/479,016; 08/899,905; 08/942,794, or corresponding PCT application PCT/US96/09511, each of which is incorporated herein by reference in its entirety. Briefly, the cells are extracted by dissociation, such as by enzymatic dissociation with collagenase alone or in conjunction with DNase, by mechanical dissociation in a blender, by teasing with tweezers, using mortar and pestle, cutting into small pieces using a scalpel blade, and the like. With respect to liquid tumors, blood or bone marrow samples may be collected and tumor cells isolated by density gradient centrifugation.

The tumor cells of the present invention may be live, attenuated, or dead (i.e., non-proliferating) cells; they may be intact; they may or may not exclude Trypan blue. Tumor cells incapable of growth and division after administration into the subject, such that they are substantially in a state of no growth, are preferred for use in the present invention. It is to be understood that “cells in a state of no growth” means live cells that will not divide in vivo. Conventional methods of rendering cells incapable of division are known to skilled artisans and may be useful in the present invention. For example, cells may be irradiated prior to use. Tumor cells may be irradiated to receive a dose of about 2500 cGy to prevent the cells from multiplying after administration. Alternatively, haptenization can render the cells incapable of growth.

The tumor cells should preferably originate from the same type of cancer as that to be treated, and are even more preferably syngeneic (e.g., autologous or tissue-type matched). For purposes of the present invention, syngeneic refers to tumor cells that are closely enough related genetically that the immune system of the intended recipient will recognize the cells as “self”, e.g., the cells express the same or almost the same complement of MHC molecules. Another term for this is “tissue-type matched.” For example, genetic identity may be determined with respect to antigens or immunological reactions, and any other methods known in the art. A syngeneic tumor cell can be created by genetically engineering a tumor cell to express the required MHC molecules.

Preferably the cells originate from the type of cancer which is to be treated, and, more preferably, from the same patient who is to be treated. The tumor cells may be, but are not limited to, autologous cells dissociated from biopsy or surgical resection specimens, or from tissue culture of such cells. Nonetheless, allogeneic cells and stem cells are also within the scope of the present invention.

### Tumor Cell Membranes

The isolated, modified tumor cell membranes of the present invention are prepared from mammalian, preferably human, tumor cells. In one embodiment of the invention, tumor cell membrane are isolated from a tumor of an animal, e.g., from a feline, canine, equine, bovine, or porcine family. Isolation and preparation of haptenized tumor cell membranes is described in U.S. patent application Ser. No. 08/479,016, filed Jun. 7, 1995 and U.S. patent application Ser. No. 90/025,012, filed Feb. 17, 1998.

The tumor cells from which membranes are isolated may be live, attenuated, or killed cells. Tumor cells rendered incapable of growth and division prior to administration into the patient, such that the cells are substantially in a state of no growth, can be used in the present invention. Alternatively, tumor cell membranes may also be isolated from tumor cells capable of in vivo growth and division, since the membranes by themselves cannot multiply. Preferably, in such a case, the tumor cell membrane preparation is not contaminated with tumor cells capable of multiplying in vivo.

As with tumor cells, tumor cell membranes are preferably isolated from the tumor cells of the same type of cancer as that to be treated. For example, membranes to be used for treating ovarian cancer are isolated from ovarian cancer cells. Preferably, the tumor cells originate from the same subject who is to be treated. The tumor cells are preferably syngeneic (e.g. autologous), but may also be allogeneic to that subject. There may be genetic identity between a particular antigen on the tumor cell used as a membrane source and an antigen present on the patient's tumor cells. The tumor cells may be, but are not limited to, cells dissociated from biopsy specimens or from tissue culture. Membranes isolated from allogeneic cells and stem cells are also within the scope of the present invention.

Tumor cell membranes may include all cellular membranes, such as outer membrane, nuclear membranes, mitochondrial membranes, vacuole membranes, endoplasmic reticular membranes, golgi complex membranes, and lysosome membranes. In one embodiment of the invention, more than about 50% of the membranes are tumor cell plasma membranes. Preferably, more than about 60% of the membranes consist of tumor cell plasma membranes, with more than about 70% being more preferred, 80% being even more preferred, 90% being even more preferred, 95% being even more preferred, and 99% being most preferred.

Preferably, the isolated membranes are substantially free of nuclei and intact cells. For example, a membrane preparation is substantially free of nuclei or intact cells if it contains less than about 100 cells and/or nuclei in about 2×10⁸ cell equivalents (c.e.) of membrane material. A cell equivalent is that amount of membrane isolated from the indicated number of cells. An isolated tumor cell membrane which is substantially free of cells and/or nuclei may contain lymphocytes and/or lymphocyte membranes.

Preferably, the isolated tumor cell membranes are the outer cell membranes, i.e., tumor cell plasma membranes. The membrane preparation of the invention may contain the entire outer membrane or a fraction thereof. An isolated membrane of the invention, preferably including a fraction of the outer membrane, contains an MHC molecule fraction and/or a heat shock protein fraction. The size of the membrane fragments is not critical.

Allogeneic tumor cell membranes may also be used in the methods of the present invention with syngeneic (e.g. autologous) antigen presenting cells. This approach permits immunization of a patient with tumor cell membranes originating from a source other than the patient's own tumor. Syngeneic antigen-presenting cells process allogeneic membranes such that the patient's cell-mediated immune system may respond to them.

A tumor cell membrane (modified or unmodified) as referred to in this specification includes any form in which such a membrane preparation may be stored or administered, such as, for example, a membrane resuspended in a diluent, a membrane pellet, or a frozen or a lyophilized membrane.

The tumor cell membranes can be obtained from haptenized cells, or may be haptenized after extraction from the cells using the techniques described infra.

Tumor cell membranes are prepared from tumor cells, e.g., obtained as described above, by disrupting the cells using, for example, hypotonic shock, mechanical dissociation and enzymatic dissociation, and separating various cell components by centrifugation. Briefly, the following steps may be used: lysing tumor cells, removing nuclei from the lysed tumor cells to obtain nuclei-free tumor cells, obtaining substantially pure membranes free from cells and nuclei, and coupling the tumor cell membranes to a hapten to obtain hapten-modified tumor cell membranes. Membrane isolation may be conducted in accordance with the methods of Heike et al. J. Immunother. Emphasis Tumor Immunol. 1994; 15:165-175

In one embodiment of the invention, intact cells and nuclei may be removed by consecutive centrifugation until membranes are substantially free of nuclei and cells, as determined microscopically. For example, lysed cells may be centrifuged at low speed, such as for example, at about 500-2,000 g for about five minutes. The separation procedure is such that less than about 100 cells or nuclei remain in about 2×10⁸ cell equivalents (c.e.) of membrane material. The retrieved supernatant contains membranes which, for example, may be pelleted by ultracentrifugation at about 100,000 g for about 90 minutes. The pellet contains mainly membranes. Membranes may be resuspended, for example, in about 8% sucrose, 5 mM Tris, pH 7.6 and frozen at about −80° C. until use. Any diluent may be used, preferably one that acts as a stabilizer. To determine the quality of membrane preparation, a fraction (about 6×10⁷ c.e. membranes) may be cultured regularly. Cell colonies should not develop and cells or nuclei should not be detected by light microscopy.

Modification of the prepared cells or membranes with DNP or another hapten may be performed by known methods, e.g. by the method of Miller and Claman (J. Immunol., 1976; 117:1519) which involves a 30 minute incubation of tumor cells or membranes with a hapten under sterile conditions, followed by washing with sterile saline. Hapten-modification may be confirmed by flow cytometry using a monoclonal anti-hapten antibody.

The dissociated cells or isolated membranes may be used fresh or stored frozen, such as in a controlled rate freezer or in liquid nitrogen until needed. The cells and membranes are ready for use upon thawing. Preferably, the cells or membranes are thawed shortly before they are to be administered to a patient. For example, the cells or membranes may be thawed on the day that a patient is to be skin tested or treated.

Allogeneic tumor cell membranes or lysates may be prepared as described above. However, prior to administration to a subject the preparation may be co-incubated with syngeneic (e.g. autologous) antigen presenting cells. Syngeneic antigen-presenting cells process allogeneic membranes such that the patient's cell-mediated immune system may respond to them. This approach permits immunization of a patient with tumor cell membranes originating from a source other than the patient's own tumor. In addition, autologous tumor lysates or membranes may be incubated with allogeneic antigen presenting cells. Allogeneic tumor cell membranes and/or autologous tumor cell lysates or membranes are incubated with antigen-presenting cells for a time period varying from about a couple of hours to about several days. The membrane-pulsed antigen presenting cells are then washed and injected into the patient.

Antigen-presenting cells may be prepared in a number of ways including for example the methods of Grabbe et al. (Immunol. Today, 1995;16:117-121) and Siena et al. (Exp. Hematol., 1995;23:1463-1471). Briefly, blood is obtained, for example by venipuncture, from the patient to be immunized. Alternatively, a sample of bone marrow may be collected. Alternatively, blood leukocytes may be obtained by leukapheresis. From any of these sources, mononuclear leukocytes are isolated by gradient centrifugation. The leukocytes may be further purified by positive selection with a monoclonal antibody to the antigen, CD34. The purified leukocytes are cultured and expanded in tissue culture medium (for example, RPMI-1640 supplemented with serum, such as fetal calf serum, pooled human serum, or autologous serum). Alternatively, serum-free medium may be used. To stimulate the growth of antigen-presenting cells, cytokines may be added to the culture medium. Cytokines include but are not limited to granulocyte macrophage-colony stimulating factor (GM-CSF), interleukin 4 (IL4), TNF (tumor necrosis factor), interleukin 3 (IL3), FLT3 ligand and granulocyte colony stimulating factor (G-CSF).

The antigen-presenting cells isolated and expanded in culture, for example, may be characterized as dendritic cells, monocytes, macrophages, and Langerhans cells.

### Tumor Cell Peptides

The isolation of peptides to be used in hapten-modified anti-cancer vaccines is described in U.S. patent application Ser. No. 08/479,016, filed Jun. 7, 1995 and provisional Patent Application 60/109,622, filed Nov. 24, 1998. Both applications disclose extraction and isolation of hapten-modified peptides, which can be adapted for the present invention. Peptides can also be synthesized based on known sequences, or isolated prior to haptenization. The isolated peptides can then be modified by haptenization.

For purposes of the present invention, peptides are compounds of two or more amino acids and include proteins. Peptides will preferably be of low molecular weight, of about 1,000 kD to about 10,000 kD, more preferably about 1,000 kD to about 5,000 kD, which are isolated from a haptenized tumor cell and which stimulate T cell lymphocytes to produce gamma interferon. The peptide of the invention may be from about 8 to about 20 amino acids, preferably from about 8 to about 12 amino acids. In addition, the peptide is preferably haptenized. Peptides may be isolated from the cell surface, cell interior, or any combination of the two locations. The extract may be particular to type of cancer cell (versus a normal cell). The peptides of the present invention include but are not limited to peptides which bind to MHC molecules, a cell surface-associated protein, a peptide associated with a heat shock protein/chaperonin, a protein encoded by cancer oncogenes, or mutated anti-oncogenes. In one preferred embodiment of the invention, peptides are bound to the MHC molecules. For purposes of the present invention “a peptide equivalent” is the peptide having the same amino acid sequence as the peptide isolated from an MHC molecule, although prepared either by degradation of a protein comprising the peptide, synthesized in vitro or recombinant DNA technology.

Preferably, the peptides are derived from tumor specific antigens. There is substantial evidence that the same T-cell-defined tumor antigens are expressed by different human melanoma tumors, suggesting that transformation-associated events may give rise to recurrent expression of the same tumor antigen in tumors of related tissue and/or cellular origin (Sahasrabudhe et al., J. Immunol., 1993;151:6302-6310; Shamamian et al., Cancer Immunol. Immunother., 1994;39:73-83; Cox et al., Science, 1994;264:716; Peoples et al., J. Immunol., 1993;151:5481-5491; Jerome et al., Cancer Res., 1991;51:2908-2916; Morioke et al., J. Immunol., 1994;153:5650-5658). Examples of such antigens include, but are not limited to, MART 1/Melan A, gp-100, and tyrosinase (melanoma); MAGE-1 and MAGE-3 (bladder, head and neck, non-small cell carcinoma); HPV E6 and E7 proteins (cervical cancer); HER2/neu/c-erbB-2 (breast cancer); HER3, HER4, Mucin (MUC-1) (breast, pancreas, colon, prostate); prostate specific antigen (PSA) (prostate); and CEA (colon, breast, GI).

The cell extracts of the invention, including peptides originally isolated from MHC molecules located on tumor cell plasma membranes, have the property of stimulating T cells. For purposes of the present invention, stimulation refers to proliferation of T cells as well as production of cytokines by T cells in response to the cell extract. Proliferation of T cells may be observed by uptake by T cells of modified nucleic acids, such as but not limited to ³H thymidine, ¹²⁵IUDR (iododeoxyuridine); and dyes such as 3-(4,5-dimethylthiazol-2-yl)-2,5-diphenyltetrazolium bromide (MTT) which stains live cells. In addition, production of cytokines such as but not limited to γ-interferon (INFγ), tumor necrosis factor (TNF), and interleukin-2 (IL-2) may be tested. Production of cytokines is preferably in an amount greater than 15 picograms/ml, more preferably about 20 to about 30 picograms/ml, even more preferably about 50 picograms/ml. Alternatively, cytotoxicity assays can be used to evaluate T cell stimulation.

From the hapten-modified cells, peptides may be extracted, some of which are hapten-modified as a result of modifying the cells. Alternatively, extracted or synthetic peptides can be reacted with a hapten after isolation or synthesis. Protein extraction techniques known to those of skill in the art may be followed by antigen assays to isolate proteins or peptides effective for patient treatment. The methods of isolating cell extracts are readily known to those skilled in the art. Briefly, cancer cells are isolated from a tumor and cultured in vitro. A hapten preparation is added to the cultured cells in accordance with the method set forth above. Peptides are isolated from cells according to an established technique, e.g., the technique of Rotzschke et al., Nature, 1990;348:252, the disclosure of which is hereby incorporated by reference in its entirety. The cells are treated with a weak acid such as but not limited to trifluoroacetic acid (TFA). The cells are thereafter centrifuged and the supernatant is saved. Compounds having a molecular weight greater than 5,000 kD are removed from the supernatant by gel filtration (G25 Sepharose, Pharmacia). The remainder of the supernatant is separated on a reversed-phase HPLC column (Superpac Pep S, Pharmacia LKB) in 0.1% TFA using a gradient of increasing acetonitrile concentration; flow rate=1 ml/min, fraction size=1 ml. Fractions containing small peptides are collected by HPLC according to the method of Sambrook et al., Molecular Cloning: A Laboratory Manual, 2nd ed., Cold Spring Harbor Laboratory Press, Cold Spring Harbor, N.Y. (1989), concentrated, and frozen.

The HPLC fractions containing small peptides are screened for immunological activity, e.g., by allowing them to bind to autologous B lymphoblastoid cells which are then tested for their ability to stimulate tumor-specific T lymphocytes. T cells used for this testing are isolated from a human patient and propagated in vitro as described in International Application No. PCT/US97/15741, published on Apr. 9, 1998 (WO 98/14206). The peptides that stimulate T cells are then analyzed for their structure. For example, the peptides are sequenced using methods known in the art to determine their amino acid sequence. In one embodiment of the invention, the peptides are sequenced as a pool as described by Burrows et al. (J. NeuroSci. Res., 1997;49:107-116) and Gavin et al. (Eur. J. Immunol., 1994;24:2124-33) to determine prevailing motifs. In another embodiment of the invention, the peptides are further separated using methods known in the art, such as HPLC, as described in U.S. Pat. Nos. 5,747,269; 5,487,982; 5,827,516 and 5,820,862 and sequenced. Sequencing is performed by using Edman degradation as described in Edman and Berg, Eur. J. Biochem., 1967;80:116-132, or any modification thereof known in the art. One powerful technique for characterizing isolated peptides is mass spectrometry.

Once the sequence of the peptides isolated from the MHC molecules is known, synthetic peptides having the same sequence are synthesized and used as a vaccine alone, presented on an antigen presenting cell and/or in combination with other extracts or whole cells using the methods described above. The equivalent peptides may also be produced recombinantly or by chemical degradation of proteins containing the isolated peptides.

In another embodiment, the structure of known peptides is altered by changing at least one amino acid and the so altered peptides are tested for their ability to stimulate T cells.

### Haptenization

The tumor cells, membranes, or peptides are haptenized. For purposes of the present invention, virtually any small molecule, including peptides, that fails to induce an immune response when administered alone, may function as a hapten. A variety of haptens of different chemical structure have been shown to induce similar types of immune responses: e.g., dinitrophenyl (DNP); trinitrophenyl (TNP) (Kempkes et al., J. Immunol., 1991;147:2467); phosphorylcholine (Jang et al., Eur. J. Immunol., 1991;21:1303); nickel (Pistoor et al., J. Invest. Dermatol., 1995;105:92); and arsenate (Nalefski and Rao, J. Immunol., 1993; 150:3806). Conjugation of a hapten to a cell may preferably be accomplished by conjugation via ε-amino groups of lysine or —COOH groups. This group of haptens include a number of chemically diverse compounds: halonitrobenzenes (including dinitrofluorobenzene, difluorodinitrobenzene, trinitrofluorobenzene), N-iodoacetyl-N′-(5-sulfonic-1-naphthyl)ethylene diamine, nitrobenzene sulfonic acids (including trinitro-benzenesulfonic acid and dinitrobenzene sulfonic acid), fluorescein isothiocyanate, arsenic acid benzene isothiocyanate, and dinitrobenzene-S-mustard (Nahas and Leskowitz, Cellular Immunol., 1980;54:241). Once familiar with the present disclosure, skilled artisans would be able to choose haptens for use in the present invention.

Haptens generally include a reactive group for conjugation to a substituent on an amino acid side chain of a protein or polypeptide (e.g., a free carboxylic acid group as in the case of aspartic acid or glutamic acid; the ε-amino group of lysine; the thiol moiety of cysteine; the hydroxyl group of serine or tyrosine; the imidazole moiety of histidine; or the aryl groups of tryptophan, tyrosine, or phenylalanine). As used herein, the term “reactive group” refers to a functional group on the hapten that reacts with a functional group on a peptide or protein. The term “functional group” retains its standard meaning in organic chemistry. These reactive groups on a hapten are termed herein the “hapten reactive group”. Numerous hapten reactive groups are known, which interact with the substituents present on the side chains of amino acids that comprise peptides and proteins. Preferred examples of such reactive groups for conjugation to specific polypeptide substituents are carboxylic acid or sulfonic acid derivatives (including acid chlorides, anhydrides, and reactive carboxylic esters such as N-hydroxysuccinimide esters), imidoesters, diazonium salts, isocyanates, isothiocyanates, halonitrobenzenes, α-halocarbonyl compounds, maleimides, sulfur mustards, nitrogen mustards, and aziridines.

Functional groups reactive with primary amines. Hapten reactive groups that would form a covalent bond with primary amines present on amino acid side chains would include, but not be limited to, acid chlorides, anhydrides, reactive esters, α,β-unsaturated ketones, imidoesters, and halonitrobenzenes. Various reactive esters with the capability of reacting with nucleophilic groups such as primary amines are available commercially, e.g., from Pierce (Rockford, Ill.).

Functional groups reactive with carboxylic acids. Carboxylic acids in the presence of carbodiimides, such as EDC, can be activated, allowing for interaction with various nucleophiles, including primary and secondary amines. Alkylation of carboxylic acids to form stable esters can be achieved by interaction with sulfur or nitrogen mustards, or haptens containing either an alkyl or aryl aziridine moiety.

Functional groups reactive with aromatic groups. Interaction of the aromatic moieties associated with certain amino acids can be accomplished by photoactivation of an aryl diazonium compound in the presence of the protein or peptide. Thus, modification of the aryl side chains of histidine, tryptophan, tyrosine, and phenylalanine, particularly histidine and tryptophan, can be achieved by the use of such a reactive functionality.

Functional groups reactive with sulfhydryl groups. There are several reactive groups that can be coupled to sulfhydryl groups present on the side chains of amino acids. Haptens containing an α,β-unsaturated ketone or ester moiety, such as maleimide, provide a reactive functionality that can interact with sulfhydryl as well as amino groups. In addition, a reactive disulfide group, such as 2-pyridyldithio group or a 5,5′-dithio-bis-(2-nitrobenzoic acid) group is also applicable. Some examples of reagents containing reactive disulfide bonds include N-succinimidyl 3-(2-pyridyl-dithio)propionate (Carlsson, et al., Biochem J., 1978; 173:723-737), sodium S-4-succinimidyloxycarbonyl-alpha-methylbenzylthiosulfate, and 4-succinimidyloxycarbonyl-alpha-methyl-(2-pyridyldithio)toluene. Some examples of reagents comprising reactive groups having a double bond that reacts with a thiol group include succinimidyl 4-(N-maleimidomethyl)cyclohexane-1-carboxylate and succinimidyl m-maleimidobenzoate.

Other functional molecules include succinimidyl 3-(maleimido)propionate, sulfosuccinimidyl 4-(p-maleimido-phenyl)butyrate, sulfosuccinimidyl 4-(N-maleimidomethyl-cyclohexane)-1-carboxylate, maleimidobenzoyl-N-hydroxy-succinimide ester. Many of the above-mentioned reagents and their sulfonate salts are available from Pierce (Rockford, Ill.).

Haptens also include a hapten recognition group that interacts with antibody. The recognition group is irreversibly associated with the hapten reactive group. Thus, when the hapten reactive group is conjugated to a functional group on the target molecule, the hapten recognition group is available for binding with antibody. By selecting an appropriate hapten reactive group, antibody recognition of, and binding to, a hapten recognition group can be made independent of the functional group to which the hapten is conjugated. When this is the case, the haptens are functionally equivalent, and are said to share antibody binding features. Naturally, in cases where the recognition groups of two haptens differ chemically, the reactive groups may be the same or different, i.e., reactive with the same or different functional groups on the target molecule.

Examples of different hapten recognition groups include without limitation to dinitiophenyl, trinitrophenyl, fluorescein, other aromatics, phosphorylcholine, peptides, advanced glycosylation endproducts (AGE), carbohydrates, etc.

In a specific embodiment, the same hapten recognition group can be coupled to different amino acids through different hapten reactive groups. For example, the reagents dinitrobenzene sulfonic acid, dinitro-phenyldiazonium, and dinitrobenzene-S-mustard, all form the dinitrophenyl hapten coupled to amino groups, aromatic groups, and carboxylic acid groups, respectively. Similarly, an arsonic acid hapten can be coupled by reacting arsonic acid benzene isothiocyanate to amino groups or azobenzenearsonate to aromatic groups.

### Isolation and Haptenization of Tumor Cells

The cells may be frozen or cryopreserved according to any method known in the art, either before or after any modification to the cells (e.g., haptenization, lysis, etc.) has been made. The dissociated cells, cell membranes, or peptides may be stored frozen in a freezing medium (e.g., prepared from a sterile-filtered solution of 50 ml Human Serum Albumin (HSA) (American Red Cross) added to 450 ml of RPMI 1640 (Mediatech) supplemented with L-glutamine and adjusted to an appropriate pH with NaOH), such as in a controlled rate freezer or in liquid nitrogen until needed. The cells are ready for use upon thawing. Optionally, the cells may be washed, and optionally irradiated to receive a dose of about 2500 cGy. They may then be washed again and suspended in Hanks Balanced Salt Solution (HBSS) without phenol red and without HSA.

Alternatively, the concentration of dissociated tumor cells can be adjusted to about 5-10×10⁷/ml, or to about 5×10⁷ or 10×10⁷ cells per ml, in HBSS. The freezing medium can be a plain cell growth medium such as HBSS, or a medium or buffer complemented with HSA, sucrose, dextran, or mixtures thereof. Preferably, the freezing medium is based on HBSS and complemented with either HSA/sucrose or HSA/dextran. The cells can also be added in equal volume to chilled 2×freezing medium containing 15% dimethylsulfoxide (DMSO) and 4% human serum albumin (HSA), with or without a suitable concentration of sucrose or dextran. The final suspension of 2 to 4×10⁷ cells/ml is placed in 1.2 ml Nunc freezer vials. In preparation for freezing, the Nunc vials are transferred on ice to a Cryo-Med model 990 Biological Freezer with a model 700 Controller and a model 500 Temperature Recorder for controlled-rate freezing. Care should be taken that the temperature of the individual vials, including the monitor vial, is uniform at the beginning of the freezing process. Vials are cooled at a controlled rate of −1° C./min to a final temperature of −80° C. The vials are then transferred in liquid nitrogen to liquid nitrogen storage.

The invention contemplates any combination of different haptens for the compositions; and thus haptenization of each tumor cell preparation with any of the foregoing haptens. The following specific procedures exemplify well known haptenization procedures, but are not intended to limit the invention.

DNP modification. Modification of the prepared cells with DNP or another hapten may be performed by known methods, e.g. by the method of Miller and Clanian (J. Immunol., 1976; 117:151), incorporated herein by reference in its entirety, which involves a 30 minute incubation of tumor cells with DNFB under sterile conditions, followed by washing with sterile saline or HBSS/HSA. Since DNP modifies hydrophilic residues of MHC-bound peptides (mainly lysine ε-amino groups) (Nahas and Leskowitz, Cellular Immunol, 1980;54:241), the second hapten could advantageously be conjugated to hydrophobic residues (such as tyrosine and histidine). Such haptens, binding proteins through an azo linkage, include sulfanilic acid (SA), arsanilic acid, and phosphorylcholine.

SA modification. Modification of the prepared cells with SA can be performed by known methods. Preferably, SA haptenization of tumor comprises an optimized pH in a buffered cultured medium. Prior methods have utilized a pH of about 8.2. However, an unexpected improvement in cell yields during the haptenization process can be achieved by reducing the pH to about 7.8. An even greater improvement in cell yield occurs when the pH is lowered to about 7.2. Preferably, the pH is in a physiological range. Upper pH limits are determined by the need to avoid loss of intact tumor cells. The pH can be below 8.2 and above 6.5, preferably between 8.0 and 6.7, more preferably between 7.8 and 7.0, and even more preferably between 7.6 and 7.2, and still more preferably between about 7.4 and 7.2. In a preferred embodiment, the pH is about 7.2.

The present invention also provides for SA haptenization of tumor cells using an optimized incubation time wherein the tumor cells are exposed to SA hapten. The incubation time can be, for example, 15 minutes or less, preferably no more than 10 minutes, more preferably no more than 5 minutes. In a preferred embodiment, the incubation time period is less than 15 minutes, preferably between 12 minutes and 1 minute, more preferably between 10 minutes and 3 minutes, even more preferably between 6 minutes and 4 minutes. Most preferably, the incubation is 5 minutes. Optimally, the incubation time, while less than 15 minutes, is long enough to produce higher yield of haptenized tumor cells than the number of viable haptenized tumor cells produced with a 15 minute incubation.

It is also preferred that the incubation time for SA haptenization of tumor cells, while less than 15 minutes, is long enough so that the degree of SA modification overall, whether the cells are intact or not, is not significantly lower than the degree of SA modification produced by the 15 minute incubation time period. It is also preferred that the degree of SA modification is no more than 70% lower than the degree of SA modification produced under the 15 minute protocol. More preferably, the degree of modification is not more than 50% lower, even more preferably not more than 25% lower. Most preferably, the degree of SA modification is equal to or higher than the degree of SA modification produced by the 15 minute incubation period.

In addition, the present invention provides a method of SA haptenization of tumor cells in which an optimized incubation time is utilized in conjunction with an optimized pH to achieve increased yields of intact tumor cells. In a previous protocol, the incubation time was 15 minutes and the pH was 8.2. However, an unexpected improvement in cell viability during the haptenization process can be achieved by jointly reducing the incubation time and pH, such that the incubation time is 10 minutes and the pH is 7.8. An even greater improvement in cell viability occurs when the incubation time is reduced to five minutes and the pH is reduced to 7.2.

In addition, the method of the present invention comprises a method of SA-haptenization of tumor cells in which the buffers HBSS and PBS are utilized during the haptenization process. The buffer PBS at pH 7.2 is preferable to BBS due to the better ability of PBS to mimic physiologic conditions. PBS is also preferred due to increased haptenized tumor cell recovery when PBS is utilized.

### Mixed-Haptenized Tumor Cell Compositions

The present invention provides multi- or mixed haptenized tumor cell compositions. The proportional ratio of tumor cells carrying one hapten to the tumor cells carrying a different hapten may vary depending on the number of haptens utilized. Furthermore, the proportional ratios of differentially haptenized tumor cells may be varied to achieve a more effective immune response. Preferably, the proportional ratio of tumor cells carrying one hapten to tumor cells carrying a different hapten varies by no more than six-fold, more preferably no more than four-fold, even more preferably no more than two fold. In a preferred embodiment, the proportional ratio of tumor cells carrying one hapten to tumor cells carrying another hapten is about equivalent, e.g., a tumor cells composition of tumor cells modified with DNP and tumor cells modified with SA in about a 1:1 ratio.

### Vaccine Preparations

The compositions of the invention may be administered in a mixture with a pharmaceutically-acceptable carrier, selected with regard to the intended route of administration and standard pharmaceutical practice. Dosages may be set with regards to weight, and the clinical condition of the patient. The proportional ratio of active ingredient to carrier naturally depend on the chemical nature, solubility, and stability of the compositions, as well as the dosage contemplated. The amount of the tumor cells of the invention to be used depend on such factors as the affinity of the compound for cancer cells, the amount of cancer cells present, and the solubility of the composition. The compounds of the present invention may be administered by any suitable route, including inoculation and injection via, for example, intradermal, intravenous, intraperitoneal, intramuscular, and subcutaneous routes.

In one embodiment of the invention, the composition comprises a vaccine comprising about 1×10⁶ to about 25×10⁶, more preferably about 2.5×10⁶ to about 7.5×10⁶, haptenized tumor cells or tumor cell equivalents (c.e.) suspended in a pharmaceutically acceptable carrier or diluent, such as but not limited to Hanks solution, saline, phosphate-buffered saline (PBS), and water. In another embodiment, the tumor cell vaccine comprises from about 5×10⁴ to about 5×10⁶ cells or cell equivalents, for example, 5×10⁴, 5×10⁵, or 5×10⁶ tumor cells or c.e. The number of cells preferably reflects the total number of cells, including both trypan-excluding and non-excluding cells. The composition may be administered by intradermal injection into 3 contiguous sites per administration on the upper arms or legs, excluding limbs ipsilateral to a lymph node dissection.

### Formulations

The formulations according to the invention may be prepared in various ways. The different components may be mixed together, and then added to haptenized tumor cells. It is also possible to mix one or several of the components with the haptenized tumor cells and then add the remaining component(s). The preparation of the formulation and its addition of the haptenized tumor cells are preferably performed under sterile conditions.

The respective proportions of the components of the media according to the invention may be adapted by persons skilled in the art.

Generally, HSA will be added to an appropriate buffered cell culture medium. “Human serum albumin” or “HSA” refers to a non-glycosylated monomeric protein consisting of 585 amino acid residues, having a molecular weight of about 66 kD. Its globular structure is maintained by 17 disulfide bridges, which create a sequential series of 9 double loops (Brown, “Albumin structure, function and uses”, Rosenoer, V. M. et al. (eds.), Pergamon Press: Oxford, pp. 27-51, 1977). The genes encoding for HSA are known to be highly polymorphic, and more than 30 apparently different genetic variants have been identified by electrophoretic analysis (Weitkamp, L. R. et al., Ann. Hum. Genet., 1973;37:219-226). The HSA gene comprises 15 exons and 14 introns corresponding to 16,961 nucleotides from the putative mRNA “capping” site up to the first site of addition of poly(A).

In its essence, a buffered cell culture medium is an isotonic buffered aqueous solution, such as phosphate buffered saline, Tris-buffered saline, or HEPES buffered saline. In a preferred embodiment, the medium is plain Hank's medium (no phenol red), e.g., as sold commercially by Sigma Chemical Co. (St. Louis, Mo., USA). Other tissue culture media can also be used, including basal medium Eagle (with either Earle's or Hank's salts), Dulbecco's modified Eagle's medium (DMEM), Iscove's modified Dulbecco's medium (IMDM), Medium 199, Minimal Essential Medium (MEM) Eagle (with Earle's or Hank's salts), RPMI, Dulbecco's phosphate buffered salts, Earle's balanced salts (EBSS), and borate buffered solution (BBS). These media can be supplemented, e.g., with glucose, Ham's nutrients, or HEPES. Other components, such as sodium bicarbonate and L-glutamine, can be specifically included or omitted. Media, salts, and other reagents can be purchased from numerous sources, including Sigma (St. Louis, Mo., USA), Gibco IBRL (Carlshad, Calif.), Mediatech (Herdon, Va.), and other companies. For use in humans, an appropriate medium is pharmaceutically acceptable.

Preferably, a formulation of whole, viable cells comprises an optimized HSA concentration in a buffered cultured medium, preferably HBSS (Hank's Balanced Salt Solution) In a specific embodiment, the final concentration of HSA is about 1.0% in HBSS. However, an unexpected improvement in cell viability can be achieved using at least about 0.25% HSA, a greater improvement in cell viability with 0.3% HSA (as compared to 0.1% HSA), and an even greater improvement is possible using at least about 0.5% HSA. Upper limits to the concentration are determined by the need to avoid contaminants that may be present in naturally-derived HSA, or alternatively to avoid allergic reactions to recombinant HSA. Preferably, the concentration of HSA in a formulation of the invention is no more than about 10%. More preferably, the concentration is less than or equal to about 5% and, more preferably still, less than or equal to about 2%.

Also, a composition or formulation of the invention may contain other components in addition to HSA to further stabilize the haptenized tumor cells. Examples of such components include, but are not limited to, carbohydrates and sugars, such as dextrose, sucrose, glucose, and the like, e.g., at a 5%-10% (preferably 7%) concentration; medium to long chain polyols, such as glycerol, polyethylene glycol, and the like, e.g., at 10% concentration; other proteins; amino acids; nucleic acids; chelators; proteolysis inhibitors; preservatives; and other components. Preferably, any such constituent of a composition of the invention is pharmaceutically acceptable.

### Fixation of Haptenized Cells

The haptenized tumor cells of the composition of the present invention may be fixed with ethanol to increase the stability of the composition to allow time for shipping and testing for quality control. The method of fixation that may be utilized with the method of the present invention is described in provisional U.S. application Ser. No. 60/354,094, filed Feb. 1, 2002. The application discloses the utilization of ethanol to stabilize cells. The method may be utilized before or after haptenization of the tumor cells.

In brief, ethanol preservation preferably involves the following. Tumor cells suspended in a suitable medium, such as, but not limited to, HBSS, and are kept on ice, at about 0° C. to 10° C., or at about 4° C. Optionally, the medium contains HSA at a concentration of, for example, 1% (weight by volume). Next, ethanol is added to the cells at a suitable final concentration (see below). In one embodiment, 3 ml of ice-cold ethanol solution are added per each ml of tumor cell suspension. The ethanol can be added to each tube while vortexing at low speed. The tubes are thereafter incubated in the presence of ethanol. Suitable incubation time and temperature can be determined experimentally for different tumor cell preparations. For example, it has been found that a 10 minute incubation at 4° C. is suitable for bihaptenized cells (see Examples 1-3). The cells are thereafter pelleted by centrifugation, e.g., by spinning at 1100 RPM for 7 minutes. The supernatant is aspirated to remove the ethanol-containing supernatant, and the cells washed in medium. For example, 5×10⁶ cells can be resuspended in 10 ml HBSS +1% HSA, and pelleted again by spinning at 1100 RPM for 7 minutes. This washing procedure can be repeated if necessary. After washing, the cells are pelleted, the supernatant aspirated, and the cells resuspended in the desired medium. For example, 5×10⁶ cells can be resuspended in 2 ml Hanks +1% HSA (se also “Formulations”, below). Preferably, the cells are stored in a medium suitable for administration to a subject.

### Adjuvant

In preferred embodiment, a tumor cell composition may be administered with an immunological adjuvant. The term “adjuvant” refers to a compound or mixture that enhances the immune response to an antigen (Hood et al., Immunology, Second Edition, 1984, Benjamin-Cummings: Menlo Park, Calif., p. 384). While commercially available pharmaceutically acceptable adjuvants are limited, representative examples of adjuvants include Bacille Calmette-Guerin (BCG) the synthetic adjuvant QS-21 comprising a homogeneous saponin purified from the bark of Quillaja saponaria and Corynebacterium parvum (McCune et al., Cancer, 1979;43:1619). Other adjuvants include Complete and Incomplete Freund's Adjuvant, mineral oils, surface active substances such as lipolecithin, pluronic polyols, polyanions, peptides, and oil or hydrocarbon emulsions.

It will be understood that the adjuvant is subject to optimization. In other words, the skilled artisan can engage in experimentation that is no more than routine to determine the best adjuvant to use.

### Immunostimulants and Combination Therapies

The haptenized tumor cell compositions may be co-administered with other compounds including but not limited to cytokines such as IL-2, IL-4, INFγ, IL-12, Il-15, IL-18, and GM-CSF. The tumor cells and extracts of the invention may also be used in conjunction with other cancer treatments including but not limited to chemotherapy, radiation therapy, immunotherapy, and gene therapy.

### EXAMPLES

The following examples illustrate the invention, but are not limiting thereof.

### Example 1

### Preparation of Multi-Haptenized Tumor Cell Vaccine

Four haptens disclosed in previous publications are dinitrophenyl (DNP), sulfanilic acid, arsanilic acid, and phosphorylcholine. Arsanilic acid, an arsenic-containing compound, is not suitable for clinical studies. Sulfanilic acid (SA) is safe in small quantities and is preferable to phosphorylcholine because it does not require extensive chemical modification (see below).

### Materials and Methods

Thawing and washing of cells. The procedures used here have been described extensively (see, e.g., U.S. Pat. No. 5,290,551 and PCT Publication Nos. WO 96/40173 and WO 00/38710). Cryogenic tubes containing frozen tumor cells in the haptenization process were quickly transferred, on ice, to a water bath. The frozen cells were thawed rapidly and removed from the water bath immediately preceding the melting of the last ice crystals in the cell mixture. Dimethylsulfoxide (DMSO) was diluted in “Wash and Thaw” solution. For each milliliter of the initial volume of the thawed cell mixture, aliquots of DMSO (0.05, 0.1, 0.2, 0.4, 0.8 ml) were added at 30 second intervals. The mixture was constantly swirled in between the additions of the DMSO. Thereafter, the cell mixture was allowed to sit at room temperature for 5 minutes. 10 ml of “Wash and Thaw” solution was added to the cell mixture.

The cells were then pelleted by centrifugation of the mixture at 1100 RMP for 7 minutes. The supernatant was removed and the pellet resuspended in 10 ml of HBSS (without HSA). The pellet resuspension mixture was then centrifuges at 100 RPM for 7 minutes. The supernatant was removed and the cell pellet was resuspended in 2 ml of HBSS (without HSA). The cells were then counted. The cell suspension was divided into two 1 ml aliquots. The aliquoted cell sample were labeled to reflect the hapten with which the cell samples were going to be haptenized, e.g., “SA” or “DNP”. Until the commencement of an haptenization procedure, the cells mixtures were stored at 4° C.

Haptenization with DNP. The cell mixture in one of the aliquot tubes, as described above, was haptenized with DNP. HBSS (without HSA) added to the “DNP” aliquot until the cell concentration of 5×10⁶ cells (intact TC+LY+dead)/ml was reached. 0.1 ml of DNFB solution was added to the for each 1.0 ml of cell mixture and thoroughly mixed with the cells. The cells and DNFB were incubated for 30 minutes at room temperature with gentle mixing every 10 minutes. After the 30 minute incubation period the haptenization reaction was stopped by the addition of 0.5 ml of 25% HSA/HBSS solution to the cell mixture. The cell mixture was immediately mixed. The DNP haptenized cells were pelleted by centrifugation at 1100 RMP for 7 minutes. The cells were then washed, centrifuged as above and washed again with a solution of 1.0% HSA/HBSS solution. The cells were then centrifuged as at 1100 RPM for 7 minutes.

Haptenization with SA. The cell mixture in one of the aliquot tubes, as described above, was haptenized with SA according to the following protocol.

Sulfanilic acid (SA) was converted to a diazonium salt by adding a saturating amount of sodium nitrite. For example, ice-cold, sterile filtered (0.2 μm), 10% sodium nitrite solution was added, dropwise, to a SA solution of 100 mg of anhydrous SA dissolved in 10 ml of 0.1 N HCl until saturation is reached. The saturation point corresponds approximately to a final concentration of a sulfanilic acid diazonium salt of about 40 mM. The SA diazonium salt solution was sterile filtered (0.2 μm membrane) and stored at 4° C. for no more than 7 days.

The SA diazonium salt solution was diluted 1:8 (v/v) in HBSS (without HSA). The pH was adjusted to 7.2 by dropwise addition of 1N NaOH. The SA diazonium salt/HBSS solution is then sterilized by filtration (0.2 μm membrane).

The thawed and washed cell mixture, prepared as described below, was pelleted by centrifugation at 1100 RPM for 7 minutes at 300 g. and the supernatant was removed. The pelleted cells were resuspended in enough diazonium salt/HBSS solution until a concentration of 5×10⁶ cells (TC+LY+dead)/ml was reached. The cell mixture was then incubated for 5 minutes at room temperature. After the 5 minute incubation period, the haptenization reaction was stopped by the addition of 0.5 ml of a 25% HSA/HBSS solution to the cell mixture.

Formulation of multihaptenized composition. The pelleted SA and DNP haptenized cells were resuspended in enough 1% HSA/HBSS to render the tumor cell concentration 1×10⁶ cells/ml. The cell concentration was determined by microscopic counting of a wet preparation. The results were confirmed by flow cytometry.

The SA-haptenized and DNP haptenized cells were mixed in about equal amounts to create a multi-haptenized tumor cell composition. The volumes were be adjusted to acquire the particular vaccine dosages desired. An example of the volumes and dosages used is listed below in TABLE 1.

TABLE 1

| Vaccine Dose | Volume of DNP Cells | Volume of SA Cells |
|---|---|---|
| 10 × 10⁶ | 5 ml | 5 ml |
| 5 × 10⁶ | 2.5 ml | 2.5 ml |
| 2.5 × 10⁶ | 1.25 ml | 1.25 ml |
| 1.25 × 10⁶ | 0.625 ml | 0.625 ml |

Detection of SA-Haptenized Tumor Cells. SA modification of melanoma cells was demonstrated by detection of haptenized cells by ELISA. Melanoma cells, dissociated from metastases and cryopreserved as described above, were left unmodified or modified with SA (as described above). The unmodified and SA-haptenized cells were fixed to the wells of micro-assay plates as described in provisional U.S. Patent Application Ser. No. 60/312,629, filed Aug. 15, 2001. ELISA was preformed as described in U.S. Patent Application Serial No. 60/312,629 with affinity-purified anti-SA ascites fluid. The positive control consisted of SA-modified bovine serum albumin (BSA) (Sigma, Co., St. Louis, Mo). The negative control consisted of DNP-BSA. The DNP-modified BSA was made by the addition of DNFB to albumin.

### Results

The multi-haptenized mixture may be tested to confirm the relative proportions of the different haptenized cells in a sample mixture. ELISA and flow cytometry were used to quantify and confirm the relative amounts of the differentially haptenized cells. Unmodified melanoma cells were used as controls.

The multi-haptenized cells were pelleted by centrifugation at 1100 RPM for 7 minutes. The supernatant was removed and the pelleted cells were resuspended in 0.15 ml of 1.0% HSA/HBSS solution. The cell suspension was stored at 4° C. until administered

### Example 2

### Testing of a Multi-Haptenized Vaccine

Theoretical considerations and experimental data provide a strong rationale for immunizing patients with tumor cells in which some are modified with DNP and others with SA. This “multi-haptenized” vaccine can be immunologically more potent and clinically more effective. Moreover, because it can be fixed with a low concentration of ethanol, it will more readily meet current regulatory requirements.

### Materials and Methods

Multi-haptenization. In this example, melanoma cells are modified with DNP (according to Example 1), and then mixed with an equal amount of melanoma cells modified with SA (according to Example 1), such that the proportional ratio of SA-haptenized cells to DNP-haptenized cells is about 1:1. The final product is analyzed to determine the relative amounts of DNP haptenized cells to SA haptenized cells, e.g. utilizing flow cytometry methods as described above. The haptenized cells may be fixed with ethanol as described above. In addition, functional tests of hapten modification are performed to determine the autologous multi-haptenized tumor cells elicit DTH in patients who have been immunized with mono-haptenized (either DNP or SA) vaccine, and multi-haptenized cells are tested for their ability to stimulate T cell responses in PBL obtained from patients after immunization with mono-haptenized vaccine.

Clinical Trials. Provided are positive results are achieved for multi-haptenized cells in the above quality control tests, clinical trials are undertaken to determine their immunogenicity and toxicity. Patients with surgically incurable melanoma or chemotherapy-refractory ovarian cancer, from which ample tumor tissue can be obtained, are included in the study. Except for the haptenization step, the tumor processing and vaccine preparation are identical to that of DNP-modified cells.

Cells are prepared and haptenized as described above, or as described previously (U.S. Pat. No. 5,290,551; U.S. patent applications Ser. No. 08/203,004; Ser. No. 08/475,016; and Ser. No. 08/899,905;) with the haptens SA and DNP. After haptenization and washing, the cells are suspended in HBSS supplemented with 1% HSA and stored at 4° C.

A preferred dosage-schedule for DNP-vaccine may be used (see, U.S. Patent Application No. 60/084,081, filed May 4, 1998). The vaccine (about 2.5-7.5×10⁶ trypan-blue excluding (i.e., live) tumor cells mixed with BCG) is injected intradermally on the upper arm (excluding arms ipsilateral to a lymph node dissection). The schedule consists of weekly administrations for about 6 weeks. Cyclophosphamide 300 mg/M² is administered intravenously about 3 days before the first vaccine injection.

The study endpoints are the evaluations of immunological responses and toxicity. Toxicity is anticipated to be limited to local vaccine reactions as with the DNP-vaccine. The major immunological parameter is DTH to SA-modified autologous tumor cells; this may be tested pre-treatment and about 2½ weeks after the last vaccine administration. Establishing, with 95% confidence interval, a positive DTH response (more than or equal to 5 mm mean diameter of induration) in at least 50% of the patients would require about 10 to about 20 patients (more than or equal to 9 out of 10 positive, or more than or equal to about 15 out of 20 positive). DTH to unmodified autologous tumor cells is measured a control. If equal to or more than 11 out of 20 patients develop a positive DTH to unmodified cells, it can be concluded, with 95% confidence interval, that the response rate is at least 30%, which is similar to what has been observed with DNP-modified vaccines. Also the development of tumor inflammatory response is studied. Negative controls include: diluent (HBSS balanced salt solution +1% HSA), autologous peripheral blood lymphocytes (PBL), and autologous PBL coated with collagenase and DNAse (the enzymes used for tumor cell processing).

Patients will be skin-tested with both mono- and multi-haptenized autologous tumor cells. For this study, a positive result would require post-vaccine DTH responses of more than or equal to 5 mm induration to DNP-modified, SA-modified, and dual-haptenized cells in more than or equal to about 15 out of 20 patients. More than 11 out of 20 patients (lower end of 95% confidence limit is about 30%) may develop positive DTH to unmodified autologous unmodified (i.e., un-haptenized) tumor cells as well.

Tests of in vitro T cell function (as described above) can be performed. If positive DTH responses to mixtures of SA and DNP modified tumor cells are observed, the T cell responses to PBL obtained post-treatment exhibit T cells will be evaluated by established methods using INFγ production and proliferation as the primary indicators of T cell response (see, U.S. patent application Ser. No. 08/479,016, filed Jun. 7, 1995).

In addition, a vaccine consisting of cells modified with phosphorylcholine (PC) can be made and evaluated. A methodology for PC coupling has been developed (Jang et al., Eur. J. Immunol., 1991;21:1303), which involves the conversion of PC to p-nitrophenyl-phosphorylcholine (Chesbro and Metzger, Biochemistry, 1972; 11:766).

In another clinical study, patients with surgically incurable melanoma, from whom adequate amounts of tumor tissue can be harvested, are included. Since preliminary data have indicated that small lung metastases are most likely to respond to haptenized tumor cell vaccine, a separate trial including only this subset of patients may be performed. Additional studies in patients with e.g., chemotherapy-refractory ovarian cancer, or melanoma patients with bulky, resectable lymph node metastases, can be conducted.

A phase II trial may employ the standard two-stage design of Simon (Controlled Clin. Trials, 1989;10:1). For example, initially, 13 patients are treated and if at least one partial response (defined by standard clinical criteria) is observed, the number of patients is expanded to 27. The dosage-schedule of the vaccine and the immunological endpoints are similar to that used in the single hapten studies.

In addition, in a preferred embodiment, a phase II trial may be performed utilizing the lowest dose that is found to be immunologically effective in the phase I trial is conducted. The immunological basis of a newly discovered phenomenon—the importance of the timing of a vaccine “induction” dose, is investigated. The hypothesis that the administration of an induction dose timed optimally with administration of low dose cyclophosphamide results in selective depletion of suppressor T cells that would otherwise down-regulate or abrogate the anti-tumor immune response is tested. Peripheral blood lymphocytes are obtained from patients at various time points and assayed for the presence of suppressor cells. It is then determined whether such suppressor cells have a characteristic phenotype, CD4⁺CD25⁺ with co-expression of CTLA4, and whether upon stimulation they produce the immunoregulatory cytokine, IL10. Finally, the ability of the suppressor cells to down-regulate in vitro T cell responses to alloantigens, hapten-modified tumor cells, and unmodified tumor cells, is tested. These studies provide insights into the immunobiology of human cancer vaccines and assist in the development of more effective immunotherapy strategies.

### Example 3

### Preparation of SA-Haptenized Tumor Cells

The technique for SA conjugation, in the present invention was developed to improve the yield of haptenized tumor cells at the end of the SA-haptenization process. Although previous methods had utilized borate buffer at pH 8.2 and a 15 minute SA haptenization incubation process, it was surprisingly determined that a pH in a range close to physiological pH yielded better yields of intact melanoma cells after haptenization while maintaining acceptable levels of haptenization. It was further determined that the incubation time could be reduced from 15 to 5 minutes with the surprising result of increasing intact cell yield with an useful degree of haptenization. In addition, it was determined that HBSS and PBS could be utilized in the protocol instead of borate buffer. Furthermore, it was surprisingly discovered that reducing the incubation time and the pH during the haptenization process increased the yield of intact cells while maintaining an useful degree of haptenization.

### Materials and Methods

Melanoma cells were SA-haptenized according to the methods described above with variations in the type of buffer, the pH of the buffer and time of incubation of the tumor cells with the SA hapten to determine the effect upon the SA-haptenization of the tumor calls and the intact cell yield of SA-haptenized tumor cells.

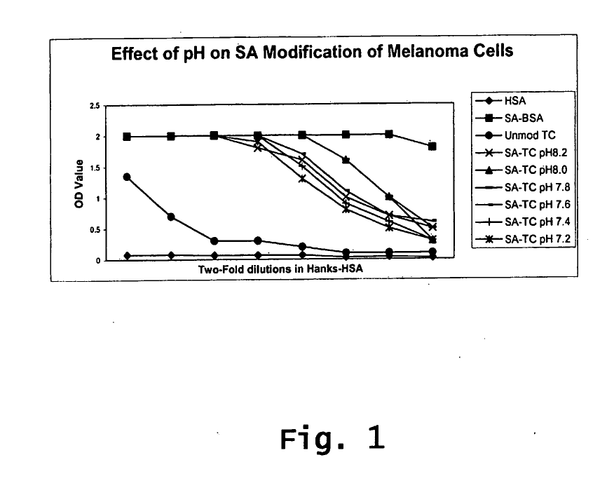

Effect of pH on the SA-modification of cells. SA ELISA experiment, as shown in FIG. 1 was run to determine the degree of SA modification of melanoma cells when the SA haptenization procedure was run with variations in the pH of the buffer. In FIG. 1, the effect of changing the pH during the haptenization process on the SA modification of melanoma cells is demonstrated. Acceptable levels of SA haptenization were found as the pH of the buffering solution (BBS) was dropped from pH 8.2 to pH 7.2 when the incubation time was set at 5 minutes. The positive control is SA-BSA and the negative controls are HSA and unmodified human melanoma cells (Unmod TC).

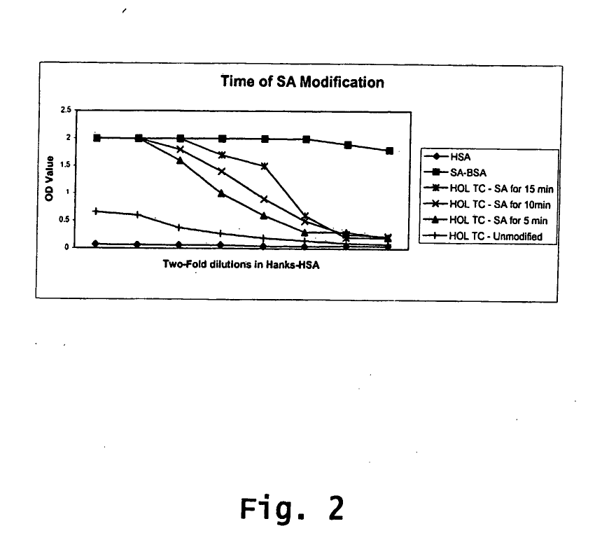

Time of SA modification. An SA ELISA experiment, as shown in FIG. 2, was run to analyze the effect of the length of time of incubation period on the degree of SA modification of melanoma cells produced by the SA haptenization procedure. The time of incubation was varied between 15 and 5 minutes and the pH was set at 8.2. In FIG. 2, the effect of changing the length of the period of incubation during the haptenization process on the SA modification of melanoma cells is demonstrated. FIG. 2 demonstrates that there is little difference in the modification when the incubation time is reduced from 15 to 5 minutes. The positive control is SA-BSA and the negative controls are HSA and unmodified human melanoma cells (HOL TC-Unmodified).

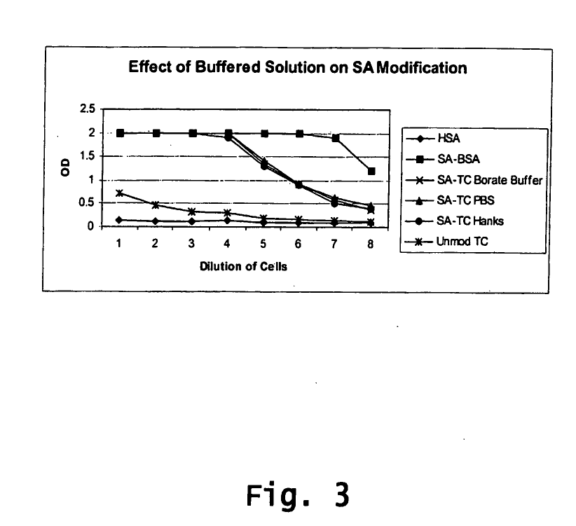

Effect of Buffered Solution on SA-modification of cells. An SA ELISA experiment, as shown in FIG. 3, was run to analyze the effect of buffering solutions on the degree of SA modification of melanoma cells produced by the SA haptenization procedure. The SA haptenization procedure was run at pH 7.2 and the incubation time was set at 5 minutes. The buffers PBS, BBS and HBSS were tested. In FIG. 3, the effect of buffered solution on the SA modification of tumor cells during the SA haptenization process run at pH 7.2 with an incubation time of 5 minutes is demonstrated. The positive control is SA-BSA and the negative controls are unmodified tumor cells (Unmod TC) and HSA. The buffered solutions, HBSS, PBS and BBS were tested.

The Effect of Buffered Solution, pH and Incubation Time on Intact Cell Yield The effect of changes in the incubation time, buffering solution and pH of the SA-haptenization protocol on the yield of SA-haptenized intact cells was examined. A series of SA haptenization were run utilizing melanoma cells with variations in the pH, time and buffer solution utilized in the haptenization process. Unmodified tumor cell controls were run under parallel conditions, save for the addition of the hapten. The cell yields were determined by flow cytometry methods and controls known in the art.

### Intact Cell Yield Results

The results of the experiments are tabulated in TABLE 2. Reducing the pH from 8.2 to 7.2 results in an increased cell yield, while not affecting the level of SA modification. This effect increases when the incubation time in the SA haptenization process is reduced from 15 to 5 minutes. In TABLE 2, the results show that there is little difference in cell yields when HBSS, PBS and BBS is used as a buffer.

TABLE 2

| | | | | Unmodified | | | SA-Modified | | | |
|---|---|---|---|---|---|---|---|---|---|---|
| Sample | pH | time | Medium | TC | LY | Dead | TC | LY | Dead | TC Yield After SA |
| DU | 8.2 | 5 | BBS | 6.2 | 36.2 | 8 | 3.4 | 18.4 | 17.6 | 55% |
| DU | 8.2 | 10 | BBS | 6.2 | 36.2 | 8 | 1.6 | 9.4 | 13.4 | 26% |
| DU | 8.2 | 15 | BBS | 6.2 | 36.2 | 8 | 0.4 | 3.4 | 4.2 | 6% |
| HOL | 8.2 | 5 | BBS | 5.8 | 17.2 | 9.2 | 2.6 | 4.4 | 5.4 | 45% |
| HOL | 8.2 | 10 | BBS | 5.8 | 17.2 | 9.2 | 1.8 | 2.6 | 9.6 | 31% |
| HOL | 8.0 | 5 | BBS | 6.6 | 13.8 | 7 | 4.6 | 12.4 | 8.8 | 70% |
| HOL | 8.0 | 10 | BBS | 6.6 | 13.8 | 7 | 2.2 | 5 | 11.8 | 33% |
| DU | 8.0 | 5 | BBS | 6.4 | 41 | 4.8 | 2.4 | 17.6 | 5.6 | 38% |
| DU | 7.8 | 5 | BBS | 6.4 | 41 | 4.8 | 3.8 | 18.6 | 5.4 | 59% |
| DU | 7.6 | 5 | BBS | 6.4 | 41 | 4.8 | 3.6 | 18.2 | 5.4 | 56% |
| DU | 7.4 | 5 | BBS | 6.4 | 41 | 4.8 | 3.8 | 16.2 | 3.6 | 59% |
| DU | 7.2 | 5 | BBS | 6.4 | 41 | 4.8 | 4.2 | 16.6 | 4.2 | 66% |
| DIC | 7.2 | 5 | HBSS | 5.2 | 2.2 | 3.4 | 3.4 | 0.6 | 3.4 | 65% |
| DIC | 7.2 | 5 | PBS | 5.2 | 2.2 | 3.4 | 3.4 | 0.8 | 4.2 | 65% |

TC = Intact tumor cells
LY = Lymphocytes
Dead = Dead cells as determined by the uptake of trypan-blue

### Example 4

Clinical Protocol for Mixed-Haptenized, Ethanol-Treated Tumor Cell Vaccine

This Example describes a phase I-II trial of a human cancer vaccine, consisting of cryopreserved, irradiated autologous tumor cells, half of which have been modified with the hapten, dinitrophenyl (DNP) and half of which have been modified with the hapten, sulfanilic acid (SA). The study subjects are patients with stage IV melanoma (non-regional metastases) who have at least one resectable metastasis. The tumor tissue obtained is dissociated into single cell suspensions and cryopreserved. The yield of tumor cells (live+dead) should be ≧100×10⁶. After recovery from surgery, the patients receive a seven-week course of treatment. The DNP-modified and SA-modified cells are mixed in equal numbers, fixed with ethanol, aliquotted, and frozen. The vaccine is administered as follows: a) induction dose day 1, b) low dose cyclophosphamide day 8, c) starting day 11, weekly vaccine mixed with BCG for six weeks, d) booster injection of vaccine mixed with BCG at 6 months. Three dose levels of mixed haptenized vaccine are studied. Low dose cyclophosphamide is administered between the first and second vaccine injections, because of its ability to augment the development of cell-mediated immunity to tumor-associated antigens. The patients are evaluated for delayed-type hypersensitivity (DTH) to autologous tumor cells and for toxicity. The development of tumor inflammation and tumor regression is recorded.

Eligibility

Patients, ages 18 and above, have stage IV melanoma (non-regional metastases) with at least one metastasis that is resectable and an estimated survival of at least 6 months. Patients with residual metastases following surgery as well as those who are clinically tumor-free are included. The mass of excised tumor must be sufficient to obtain ≧100×10⁶ tumor cells (live+dead). Allowable metastatic sites from which tumor may be harvested include: lymph nodes, lung, liver, adrenal, and subcutaneous tissue. Metastatic sites that are not allowed are: bone, brain, or gastrointestinal tract. A sufficient number of vaccine cells have been prepared and frozen to administer a course of therapy, and vaccines must have passed lot release tests.

Surgery and Tumor Acquisition

Patients undergo surgical resection of metastases by standard techniques. The tumor tissue is hand delivered or shipped to the laboratory in sterile isotonic medium containing gentamicin 20 ug/ml and maintained at 4° C. The maximum time from tumor procurement to initiation of vaccine protocol is 6 months.

Materials for Vaccine Preparation

Banking Medium. 450.0 ml RPMI without phenol red (Sigma catalogue #R-7509); 50.0 ml Human Serum Albumin (25% solution; final concentration=2.5%), and 5.0 ml glutamine (Sigma Chemical Co., catalog #G6392). Adjust to pH to 7.2 with 5. N NaOH. Sterile filter through 0.2 u filter into sterile plastic bottle attached to filtration unit (Nalgene-Fisher catalog #09-740-25A).

Collagenase Solution (for making collagenase-coated lymphocytes for skin-testing). 100 ml Hanks+1% HA and 140 mg collagenase (Sigma catalogue #C-0130). Mix until completely dissolved. Sterile filter through 0.2 u filter.

Dinitrofluorobenzene (DNFB) Solution. (Reference: Miller and Claman, J Immunol 117:1519, 1976). Place 0.5 ml of 95% ethanol (USP grade—Pharmco Products) into a 50 ml beaker. Micropipet 65 μl of concentrated stock DNFB (Sigma D-1529) into the beaker. Mix by swirling for several minutes to get even suspension. Add 99.5 ml PBS (Mediatech Inc., catalogue #21-031-CV) and a sterile stirring bar to a 250 ml beaker—then add DNFB suspension—rinse small beaker with PBS. Cover beaker with parafilm and stir overnight in 370 water bath. Filter through 0.2 u filter set into sterile plastic bottle. Cover bottle with aluminum foil, and store at 4° C.

Enzyme Solution For Tumor Dissociation. 100 ml Wash and Thaw Solution, 140 mg collagenase (Sigma catalogue #C-0130), and gentamicin stock solution—1. ml. Mix until completely dissolved. Sterile filter through 0.2 u filter.

Ethanol Solution For Fixation. 100% ethanol (USP grade—Pharmco Products)—100 ml. Water—100 ml. Sterile filter

Gentamicin Stock Solution (100×). 1 vial of gentamicin (40 mg/ml−2. ml=80 mg), 38. ml Hanks (Sigma catalog #21-022-CV). Sterile filter through 0.2 u filter (final concentration of gentamicin=2 mg/ml).

Hanks+Gentamicin For Tumor Transport And Processing. Hanks (Sigma catalogue #21-022-CV)—500 ml, and gentamicin stock solution—5. ml. Sterile filter through 0.2 u filter.

Hanks+Gentamicin For Skin Testing. 10 ml Hanks+Gentamicin for Tumor Transport and Processing. 10 ml Hanks—mix. Sterile filter through 0.2 u syringe filter

Hanks+0.1% HSA. 500 ml Hanks, 2.0 ml Human Serum Albumin (25% solution). Sterile filter through 0.2 u filter.

Hanks+1.0% HAS. 500 ml Hanks, 20. ml Human Serum Albumin (25% solution). Sterile filter through 0.2 u filter.

Hanks+EDTA (for lymphocyte separation). 500. ml Hanks (Sigma catalogue #21-022-CV), add 0.5 g EDTA (Sigma catalogue #E-5134), and adjust pH to 7.2 with 5. N NaOH. Sterile filter through 0.2 u filter.

Sucrose Freezing Medium. Hanks balanced salt solution—60 ml, Human serum albumin (25% solution)—40 ml, Sucrose—8. g. Mix to dissolve completely. Sterile filter through 0.2 u filter. For skin testing, dispense 0.5 ml of Sucrose Freezing Medium per vial.

Sulfanilic Acid Diazonium Salt. Sulfanilic acid-anhydrous-Sigma-S-5643 (100 g), 10% Sodium nitrite—10 g sodium nitrite (Sigma S-3421), 100 ml water. Sterile filter through 0.2 u filter. Add 100 mg sulfanilic acid to 10. ml 0.1N hydrochloric acid (Sigma 210-4 (endotoxin-free)). Add ice-cold 10% sodium nitrite dropwise to sulfanilic acid—stir for 30 sec after each drop, then add droplet to starch-iodide paper until blue color appears (about 15 drops)—then stop (the final concentration of sulfanilic acid diazonium salt should be 40 mM). Sterile filter.

Wash & Thaw Solution. 500. ml Hanks, add 0.5 g EDTA, adjust pH to 7.2 with 5. N NaOH. Add 2.0 ml 25% Human Serum Albumin (final concentration=0.1%). Sterile filter through 0.2 u filter.

Tumor Processing

Briefly, cells are extracted by enzymatic dissociation with collagenase and by mechanical dissociation, frozen in a controlled rate freezer, and stored in liquid nitrogen until needed. Gentamicin 20 μg/ml is added to the tumor processing solution and washed out before the tumor cells are cryopreserved.

The tumor specimen is kept at 4° C. until processing—no more than 48 hours. Trim off and discard most of fat, connective tissue, and obviously necrotic material. Determine tumor weight. Add enough sterile Hanks+Gentamycin to cover bottom of a sterile Petri dish under the hood. Transfer the tumor tissue from the specimen container to the Petri dish. Cut off small sample of tumor (3-5 mm diameter) and place in vial with buffered formaldehyde; affix a prepared label. Mince tumor with scalpel so that pieces are 3-5 mm diameter. Pour minced tissue+liquid through sterile disposable filter set with sterilized steel screen: collect supernatant, pour into sterile tube (“TCM”). Keep at 4° C. until further processing.

Pipet appropriate amount of enzyme solution into disposable 125 ml or 250 ml flask with minced tumor pieces. Cap flask tightly and place in incubator shaker that has been pre-warmed to 37° C. Close the cover and set speed to about 350 RPM. Set timer on shaker for 30 minutes. After 30 minutes, turn off shaker and remove flask. Pipet fluid containing cell suspension into sterile mesh; transfer cell suspension to sterile 50 ml tube labeled “TCE”. Keep cell suspension at 4° C. until further processing. (The second digestion may be omitted if it appears that the only remaining tissue is connective tissue). Pipet enough enzyme solution to cover tissue pieces in disposable flask and place in incubator shaker for another 30 minutes at about 350 RPM. After 30 minutes, turn off shaker and remove flask. Pipet fluid containing cell suspension into sterile mesh; transfer cell suspension to sterile 50 ml tube labeled “TCE”. Pipet about 25 ml Hanks+Gentamycin into tumor dissociation flask; swirl briefly, then pipet as much of supernatant as possible through sterile mesh and add to “TCE” tube. Keep cell suspensions at 4° C. until further processing. Add Hanks (no gentamycin) to make volume of about 45 ml to each TCE tube.

Pellet the TCM and TCE tubes by centrifugation at 300 g (about 1100 RPM) for 7 minutes. Aspirate supernatants. Combine all resuspended TCE pellets in one 50 ml tube. Add about 45 ml Hanks (no gentamycin) and mix. Pellet the TCE tube by centrifugation at 300 g (about 1100 rpm) for 7 minutes. Aspirate supernatant and resuspend in Hanks (no gentamycin). Use at least 10 ml Hanks, but more can be added if pellet is very large. Resuspend the TCM pellet in 10 ml Hanks (no gentamycin).

Perform cell counts of TCE and TCM tubes according to Cell Counting Procedure. Following cell count, combine the TCE and TCM and label tubes as TC. Then, add enough Hanks (no gentamycin) to make volume of about 45 ml. Pellet cells by centrifugation at 300 g (about 1100 rpm) for 7 minutes. Aspirate supernatant.

23. Resuspend the cells in ice-cold Banking Medium, add the appropriate volume of 20% DMSO, and mix by inverting the capped tubes. Dispense the cell suspension into cryovials, and keep at 4° C. until ready to freeze. Freeze the cells in the programmed freezer and then place in liquid nitrogen bank. Cells should be maintained in the vapor phase of liquid nitrogen only.

Vaccine Preparation

Only if a sufficient number of mixed haptenized vaccine cells is obtained and the patient's vaccine passes lot release tests (endotoxin level <100 EU/ml, 14-day sterility testing negative), will patients be offered entry onto the study. Briefly, the vaccine consists of irradiated tumor cells, half of which have been haptenized with DNP and half with SA. The two types of haptenized cells are mixed in equal numbers, fixed with ethanol, and frozen. Melanoma cells may be admixed with variable numbers of tumor-associated lymphocytes and trace numbers of erythrocytes. The final volume of the vaccine is 0.2 ml.

The vaccine manufacturing procedure can be summarized as follows: The required number of autologous tumor cells will be thawed, washed, and divided into two aliquots. They will be irradiated to 2500 cGy. Then, one aliquot will be modified with dinitrophenyl (DNP) by the method of Miller and Claman (19) that we have used since 1988. This involves a 30-minute incubation of tumor cells with dinitrofluorobenzene under sterile conditions, followed by washing with Hanks solution. The second aliquot will be modified with sulfanilic acid (SA). The method is a modification of published procedures (Bach et al., J. Immunol., 121: 1460-1468, 1978; Sherman et al., J. Immunol., 121: 1432-1436, 1978; and Collotti et al., J. Exp. Med., 571-582, 1969). Cells are incubated for 5 minutes at room temperature with the diazonium salt of sulfanilic acid under sterile conditions, followed by washing with sterile Hanks solution. Following hapten modification, the cells are mixed 3:1 with 50% ethanol for a final concentration of 37.5%. Equal numbers of ethanol-treated DNP-modified and ethanol-treated SA-modified tumor cells will be mixed, washed, resuspended in cryopreservative (sucrose+human serum albumin) and dispensed in labeled vials. The vials are frozen by placing in a −86° freezer overnight, followed by transfer to and storage in liquid nitrogen. When a patient is ready to be treated, a vial of vaccine will be rapidly thawed, drawn up in a syringe, and injected intradermally within 20 minutes of thawing. Cryovials are thawed by placing in heating block at 37±0.5° until the contents are thawed with a few small ice crystals remaining. Each specific step of the vaccine preparation is described below.

Irradiation. Pellet cells by centrifugation at 300 g (about 1100 RPM) for 7 minutes. Aspirate supernatant and suspend pellet in 24 ml (depending on pellet size) of Hanks+0.1% HSA. Pipet the cell suspension to cryovials, about 2. ml per cryovial, and place in refrigerated block. Irradiate tumor cells in cesium irradiator to 2500 cGy (at the currently calculated dose rate of 106.3 cGy/min, the time is 23.5 minutes).

After irradiation, pipet cells into 15 ml centrifuge tubes. Add 10 ml Hanks-no HSA and mix. Pellet cells by centrifugation at 300 g (about 1100 RPM) for 7 minutes. Aspirate supernatant and resuspend pellet in 10 ml Hanks—no HSA. Perform cell count as per Cell Counting SOP, except: Do not add trypan blue. Count large and small nucleated cells.

Unmodified Skin Test Materials. Pipet 15×10⁶ tumor cells into tube labeled “ST-UN”. Pellet the “ST-UN” tube by centrifugation at 300 g (about 1100 RPM) for 7 minutes. Aspirate the supernatant and resuspend pellet in 1. ml cold (4° C.) Hanks with 1% HSA. Add 3. ml of ice-cold 50% ethanol to the “ST-UN” tube while vortexing at low speed. Incubate the tube at 4° for 10 minutes. Pellet cells by centrifugation at 300 g (about 1100 RPM) for 7 minutes. Aspirate supernatant and resuspend in 2. ml Hanks+1% HSA. Perform cell count of “ST-UN” tube by Cell Counting Procedure, except do not add trypan blue. Count only large and small cells.

Pellet “ST-UN” tube by centrifugation at 300 g (about 1100 RPM) for 7 minutes. Aspirate supernatant. Resuspend “ST-UN” pellet in volume of ml Sucrose Freezing Medium to make a concentration of 1×10⁶ large cells per 0.15 ml (6.7×10⁶/ml). Dispense “ST-UN” cells into vials, 0.15 ml/vial (3×10⁶ tumor cells/vial) Place cryovials at 4° C. until ready for freezing.

Hapten Modification. Divide remainder of tumor cell suspension into two equal aliquots. Label one tube “DNP” and the other “SA”. Pellet both cell suspensions by centrifugation at 300 g (about 1100 RPM) for 7 minutes. Aspirate supernatants. To the SA tube, add 2. ml Hanks-no HSA and keep at 4° C. until needed.

To the “DNP” tube add Hanks without albumin to bring the concentration of cells (tumor cells+lymphocytes) to 5×10⁶/ml. For each 1.0 ml of cell suspension, add 0.1 ml of DNFB solution. Mix and incubate at room temperature for 30 minutes; gently mix every 10 minutes.

While the DNP cells are incubating, dilute the diazonium salt of SA 1:8 in Hanks without albumin. Adjust the pH to 7.2 by dropwise addition of 1N NaOH (2-3 drops). Sterile filter the solution. Pellet the “SA” tube by centrifuging at 300 g (about 1100 RPM) for 7 minutes. Aspirate supernatant. Resuspend the pellet in a quantity of the diluted diazonium salt to make a cell concentration (intact TC+LY+dead) of 5×10⁶/ml. Immediately resuspend. Incubate for 5 minutes at room temperature.

As soon as the DNP and SA are finished (30 minutes and 5 minutes, respectively), stop the reactions by adding 0.5 ml of the stock solution of human serum albumin (25% solution) to the tube, capping, and mixing. Pellet cells by centrifugation at 300 g (about 1100 RPM) for 7 minutes. Wash the cells twice in Hanks +1.0% HSA.

Ethanol Treatment. After the last centrifugation, resuspend the cells in the DNP and SA tubes in 1. ml ice-cold (4o) Hanks with 1% HSA. Add 3. ml of ice-cold 50% ethanol to each tube while vortexing at low speed. Incubate the tubes at 4° C. for 10 minutes. Pellet cells by centrifugation at 300 g (about 1100 RPM) for 7 minutes. Aspirate supernatant, resuspend in 10 ml Hanks+1% HSA, and pellet by centrifugation at 300 g (about 1100 RPM) for 7 minutes. Aspirate supernatant and resuspend in 2. ml Hanks+1% HSA. Perform cell count of SA and DNP tubes by Cell Counting Procedure, except do not add trypan blue. Count large and small nucleated cells and erythrocytes.

To determine the proportion of dead cells: Add one drop of suspension from SA and DNP tubes to separate glass slides. Add one drop trypan blue to each slide. Place a cover slip over the drops. Perform a count of trypan-blue (+) and trypan blue (−) cells by counting 100 cells. Calculate the percentage of trypan-blue (+) cells and record in the batch record.

Haptenized Skin Test Materials. Remove 4×10⁶ large cells from SA tube and pipet into tube with affixed patient label and label “ST-SA”. Remove 4×10⁶ large cells from DNP tube and pipet into tube labeled “ST-DNP”. Pellet cells in both tubes by centrifugation at 300 g (about 1100 RPM) for 7 minutes. Aspirate supernatants. Resuspend each in 0.60 ml Sucrose Freezing Medium. Add 0.15 ml of ST-SA cells to each of 4 cryovials. Add 0.15 ml of ST-DNP cells to each of 4 cryovials. Place cryovials at 4° C. until ready for freezing.

Combining and Aliquotting SA and DNP-Haptenized Vaccine. Calculate number of remaining SA-modified and DNP-modified tumor cells. Mix equal numbers (maximum possible) of remaining SA-modified and DNP-modified cells in a tube with an affixed patient label. Pellet cells by centrifugation at 300 g (about 1100 RPM) for 7 minutes. Aspirate supernatant. Calculate the total number of SA-modified+DNP-modified tumor cells. Resuspend the pellet in Sucrose Freezing Medium.

Gently mix the vaccine cell suspension. Add 0.2 ml of the vaccine suspension to each of the pre-labeled “VACC”. Freeze all of the vials by placing them into Nalgene Cryo 1° C. Freezing Container with isopropanol in −86° C. freezer. Leave vials overnight. Then transfer to liquid nitrogen bank.

Pre-vaccine Skin-Testing

This is performed 2 weeks prior to beginning vaccine injections by the intradermal injection of 0.15 ml of test material on the forearm. DTH is assessed at 48h by measuring the mean diameter of induration. Patients are tested for DTH to the following materials:

1) 1.0×10⁶ autologous melanoma cells: irradiated (2500 cGy), DNP-modified, fixed

2) 1.0×10⁶ autologous melanoma cells: irradiated (2500 cGy), SA-modified, fixed

3) 1.0×10⁶ autologous melanoma cells: irradiated (2500 cGy), unmodified, fixed

4) diluent—Hanks solution with sucrose+human serum albumin (HSA)

All skin test materials will be prepared and frozen in advance of the date of testing. The standard operating procedure is appended. An aliquot of each material will be tested for sterility and endotoxin and the material will be used only if it passes both tests (no growth in 14-day sterility assay and endotoxin level <100 EU/ml).

Patients who have a negative baseline DTH reaction (<5 mm induration) to all three of the melanoma cell preparations will continue on the study to receive vaccine at one of the three study doses. Patients who have a positive baseline DTH reaction (>5 mm induration) to any of the three melanoma cell preparations will be eligible to receive vaccine only at dosage level B (0.5×10⁶ tumor cells).

Method for Skin Test Application and Measurement. After the patient has arrived, thaw a vial of each of the cellular materials. The thawed materials may be stored at 4° C. for 20 minutes prior to injection. The most proximal skin test should be at least 3 cm below the elbow crease on the ventral forearm and each injection should be separated by at least 3 cm. If one of the patient's forearms is unusable, e.g., because of post-surgical lymphedema, all skin test must be done on the same arm by using medial and lateral edges of the ventral forearm.

For each cellular skin test material, draw up the contents (0.15 ml) into a 0.5 cc Lo-Dose insulin syringe and inject intradermally, making sure that a wheal is raised by the injection. For soluble skin test materials (PPD, diluent, gentamicin) draw up 0.10 ml.

Measuring the Reactions. After 48±4 hours, inspect the skin test injection sites. Measure the diameters of erythema at each site, i.e., the longest diameter and the diameter perpendicular to this. Palpate each reaction to determine the induration. Measure the diameters of induration at each site; the longest diameter and the one perpendicular to this. A positive response is defined by mean diameter of induration ≧5 mm.

Vaccine Administration

The left arm is the site of all vaccine injections, unless the patient has had a left axillary lymph node dissection; in that case the right arm will be used for all vaccine injections. If a patient has undergone bilateral axillary dissections, the vaccine injections are made on the left upper thigh. See diagram.

| | | | | | |
|---|---|---|---|---|---|
| Vaccine #1 | ventral forearm | | Vaccine only | | |
| Vaccine #2 | dorsal | BCG-A only | BCG-A + vaccine | BCG-A + vaccine | BCG-A + vaccine |
| | upperarm | | | | |
| Vaccine #3 | dorsal | | BCG-A + vaccine | BCG-A + vaccine | BCG-A + vaccine |
| | upperarm | | | | |
| Vaccine #4 | dorsal | | BCG-B + vaccine | BCG-B + vaccine | BCG-B + vaccine |
| | upperarm | | | | |
| Vaccine #5 | dorsal | | BCG-B + vaccine | BCG-B + vaccine | BCG-B + vaccine |
| | upperarm | | | | |
| Vaccine #6 | dorsal | | BCG-C + vaccine | BCG-C + vaccine | vaccine only |
| | upperarm | | | | |
| Vaccine #7 | dorsal | | BCG-C + vaccine | BCG-C + vaccine | BCG-C + vaccine |
| | upperarm | | | | |
| Vaccine #8 | dorsal | | BCG-C + vaccine | BCG-C + vaccine | BCG-C + vaccine |
| | upperarm | | | | |

On day 1, patients are injected intradermally on the ventral forearm with mixed haptenized vaccine without added BCG. This serves as an induction dose of vaccine. Draw up the vaccine suspension (0.2 ml, tumor cells in Hanks solution with sucrose and human serum albumin) into a 0.5 cc “Lo-Dose” insulin syringe and inject intradermally into the mid ventral forearm.

On day 8+1, patients will receive cyclophosphamide 300 mg/M² as a bolus injection over 5 minutes. The rationale is based on published evidence from animal and clinical studies (Hengst et al., Cancer Res, 40: 2135-2141, 1980; Berd et al., Cancer Res, 46:2572-2577, 1986) showing that cyclophosphamide augments the development of cell-mediated immunity to tumor-associated antigens. Cyclophosphamide is reconstituted with bacteriostatic water for injection, USP, at a dilution of 20 mg of cyclophosphamide per 1 ml of water.

Three days later the patients is injected intradermally on the dorsal upper arm with vaccine mixed with BCG and this will be repeated weekly for a total of 6 weeks. The injection of vaccines #2-7 will be made into the same limb as the induction dose. Three dose ranges of mixed haptenized vaccine will be studied. The method of administration of vaccine #2-7 is as follows: Prepare BCG by reconstituting with 1.0 ml saline for injection (without preservative) according to package label. Prepare 1:10, 1:100, and 1:1,000 dilutions of the BCG in saline for injection and label as A, B, and C, respectively. After the patient has arrived, thaw a vial of mixed haptenized vaccine, checking for identifying information. Add 0.1 ml of the proper dilution of BCG (see below) to the vaccine suspension. Immediately draw up the vaccine-BCG mixture into a 0.5 cc “Lo-Dose” insulin syringe and inject intradermally into three adjacent sites, separated by about 1 cm, on the upper arm.

The administration of vaccines #2 and #6 is modified to allow a better assessment of toxicity. Vaccine #2: The administration of the vaccine is given as described. However, an additional dose of BCG will be administered to differentiate the local toxicity of BCG from the local toxicity of the vaccine-BCG combination. This is done as follows: Add 0.1 ml of BCG dilution “A” (1:10) to a sterile vial. Add 0.2 ml of saline for injection to the vial. Mix and withdraw 0.1 ml with a 0.5 cc “Lo-Dose” insulin syringe. Inject intradermally about 1 cm medial to the most medial vaccine injection site. Vaccine #6: Two-thirds of the vaccine dose will be injected with BCG and one-third without BCG. This is done as follows: Gently mix the thawed vaccine suspension and draw up 0.07 ml into a 0.5 cc “Lo-Dose” insulin syringe. Inject the vaccine intradermally into the most lateral of the three intended vaccine sites. Add 0.1 ml of the proper BCG dilution (“C”, 1:1,000) to the remainder of the vaccine suspension and inject intradermally into two sites medial to the first injection.

Booster Injections

Patients who have not exhibited tumor progression and who have not received other melanoma treatments in the interval will be given a booster vaccine at the six month point (measured from beginning the vaccine program) if sufficient cells are available. The dose and method of administration of the booster injections will be the same as vaccine #7.

Assignment of Vaccine Dose

Patients whose baseline DTH response to autologous melanoma cells was negative (<5 mm induration) are assigned to one of three vaccine dosage levels.

A=5.0×10⁴ tumor cells

B=5.0×10⁵ tumor cells

C=5.0×10⁶ tumor cells

A patient is assigned to one of these dosage levels according to the yield of mixed haptenized, fixed tumor cells obtained after vaccine production. If the yield of tumor cells is ≧2×10⁶ and <20×10⁶, the dose assignment is “A”. If the yield of tumor cells is ≧20×10⁶ and <55×10⁶, the dose assignment is “B”. If the yield of tumor cells is ≧55×10⁶, the dose assignment is “C”. The three dosage levels will be tested simultaneously. At least 6 and no more than 14 evaluable patients will be treated at each dosage level. After 14 evaluable patients have been treated at a given dosage level, subsequent patients are assigned to the next unfilled dosage level.

Patients who have a positive (≧5 mm induration) baseline DTH response to any of the melanoma cell preparations will be eligible to receive the vaccine at dosage level B only. If their vaccines had been aliquotted and frozen for dosage levels A or C, they will not receive vaccine treatment and will be discontinued from the study. A maximum of 14 patients with positive baseline DTH reactions will be treated.

BCG doses

The first dose (induction dose) contains no BCG. The second and third vaccines are mixed with 0.1 ml of a 1:10 dilution of Tice BCG (“Tice-A”). The fourth and fifth vaccines are mixed with 0.1 ml of a 1:100 dilution (“Tice-B”). The sixth and seventh and the booster vaccines are mixed with 0.1 ml of a 1:1000 dilution (“Tice-C”). The ideal vaccine reaction is an inflammatory papule with no more than small (<5 mm) central ulceration. If reactions are larger than this, the dose of BCG is further attenuated ten-fold.

Post-Vaccine Skin-Testing

This is performed by the intradermal injection of test material on the forearm, and DTH is assessed at 48 h by measuring the mean diameter of induration. Patients are tested for DTH to the following materials:

1) 1.0×10⁶ autologous melanoma cells: irradiated (2500 cGy), DNP-modified, fixed

2) 1.0×10⁶ autologous melanoma cells: irradiated (2500 cGy), SA-modified, fixed

3) 10×10⁶ autologous melanoma cells: irradiated (2500 cGy), unmodified, fixed

4) 5.0×10⁶ autologous peripheral blood lymphocytes, unmodified, fixed

5) 5.0×10⁶ autologous peripheral blood lymphocytes—coated with collagenase, fixed

6) diluent—Hanks solution with sucrose+human serum albumin (HSA)

7) gentamicin 1.0 μg in 0.1 ml Hanks solution

8) PPD intermediate

All skin test materials (with the exception of PPD, which is commercially available and approved for human testing) are prepared and frozen in advance of the date of testing. The volume of the cellular materials (#1-5) is 0.15 ml; the volume of materials #6-8 is 0.10 ml. The procedure for measuring and photographing DTH reactions is as described above.

Tumor Inflammation

Patients are evaluated clinically to determine whether they developed inflammation in superficial metastases (dermal and subcutaneous). This is defined as erythema in and/or around tumor sites that develops following vaccine treatment. Metastases that exhibit inflammation are photographed and are biopsied if possible.

Clinical Evaluation of Patients

Anti-tumor responses are documented. Only patients with measurable metastases at the time of beginning vaccine treatment are assessed for response. CT or MRI imaging are performed every 3 months until tumor progression. Standard definitions of response will be used:

Complete Response (CR): Complete disappearance of all clinically detectable disease by two observations no less than 4 weeks apart.

Partial Response (PR): A ≧50% decrease (in bidimensional lesions) or ≧30% decrease (in unidimensional lesions) in the total tumor size of the lesions (as determined by the sum of the products of the two greatest perpendicular diameters of all measurable lesions), which have been measured to determine the effect of therapy. The decrease is documented by two observations no less than 4 weeks apart. In addition, there are no appearance of new lesions or progression of any lesion.

Stable Disease (SD): A <50% decrease in bidimensional lesions or <30% decrease in unidimensional lesions (as defined above) or, a <25% increase in any individual lesions for a least 4 weeks.

Progressive Disease (PD): An increase of ≧25% of one or more measurable lesions or the appearance of any new lesion.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, are approximate and are provided for description only.

Patents, patent applications, and publications cited throughout this application are incorporated herein by reference in their entireties.

What is claimed is:

1. A composition comprising a mixture of a first and a second haptenized tumor cell preparation, wherein the haptenized tumor cell preparations
a) are differentially haptenized; and
b) originate from the same tumor type as the tumor type of a subject intended for treatment with the composition.

2. The composition of claim 1, comprising a first and a second hapten attached to functional groups of polypeptides associated with the tumor cells.

3. The composition of claim 2, wherein the functional groups are selected from an amino group, a carboxylic group, and an aromatic group.

4. The composition of claim 2, wherein the first hapten is sulfanilic acid (SA), and the second hapten is dinitrophenyl (DNP).

5. The composition of claim 1, wherein the numbers of cells in the first and second haptenized tumor cell preparations differ by no more than two-fold.

6. The composition of claim 1, wherein the numbers of cells in the first and second haptenized tumor cell preparations are about equal.

7. The composition of claim 1, comprising tumor cells rendered incapable of growth.

8. A vaccine for treating cancer comprising the composition of claim 1 and an adjuvant.

9. The vaccine of claim 8, wherein the adjuvant is BCG.

10. A method for preparing a composition for use in a cancer vaccine, which method comprises differentially haptenizing at least a first and a second fraction of a tumor cell preparation and mixing cells from the differentially haptenized fractions, wherein the tumor cell preparation originates from the same type of tumor as the tumor of a subject for whom the vaccine is intended.

11. The method of claim 10, wherein the first fraction is haptenized with a first hapten, and the second fraction is haptenized with a second hapten.

12. The method of claim 11, wherein the first and second haptens are conjugated to functional groups selected from an amino group, a carboxylic acid group, an aromatic group, a hydroxyl group, an imidazole group, and a sulfhydryl group.

13. The method of claim 12, wherein the first hapten is conjugated to an aromatic group and the second hapten is conjugated to a primary amino group or a carboxylic acid group.

14. The method of claim 13, wherein the first hapten is sulfanilic acid (SA), and the second group is dinitrophenyl (DNP).

15. The method of claim 10, wherein the numbers of cells mixed from the first and second fractions differ by no more than two fold.

16. The method of claim 15, wherein the numbers of cells mixed from the first and second fractions are about equal.

17. The method of claim 10, wherein the tumor cells have been rendered incapable of growth.

18. A method for treating cancer in a subject comprising administering a vaccine of claim 8 to the subject.

19. The method of claim 18, wherein the subject is a human.

20. A method of haptenizing a cells with sulfanilic acid, comprising the steps of:
(a) contacting an aromatic group of the cell with a sulfanilic-acid-diazonium-salt in a buffered solution at a pH lower than 8.2 to initiate a haptenization reaction;
(b) incubating the solution for less than 15 minutes, and
(c) terminating the haptenization reaction.

21. The method of claim 20, wherein the pH is between 7.0 and 7.8.

22. The method of claim 19, wherein the pH is 7.2.

23. The method of claim 20, wherein the incubating step is between 3 and 10 minutes.

24. The method of claim 23, wherein the incubating step is 5 minutes.

25. The method of claim 20, wherein the buffered solution is selected from HBSS and PBS.
